(19) United States
(12) Patent Application Publication
(10) Pub. No.: US 2010/0015104 A1
(43) Pub. Date: Jan. 21, 2010

(54) GENERATION OF ADIPOSE TISSUE AND ADIPOCYTES

(75) Inventors: John K. Fraser, San Diego, CA (US); Michael DeEmidio, San Diego, CA (US)

Correspondence Address: KNOBBE MARTENS OLSON & BEAR LLP, 2040 MAIN STREET, FOURTEENTH FLOOR, IRVINE, CA, 92614, US

(73) Assignee: Cytori Therapeutics, Inc

(21) Appl. No.: 12/375,005

(22) PCT Filed: Jul. 25, 2007
(86) PCT No.: PCT/US07/16750

§ 371 Date: Sep. 25, 2009

Related U.S. Application Data
(60) Provisional application No. 60/833,561, filed on Jul. 26, 2006.

(51) Int. Cl. A61K 35/12; C12Q 1/04; C12Q 1/02; A61P 3/00

(52) U.S. Cl. 424/93.7; 435/34; 435/29

(57) ABSTRACT

The invention provides novel methods by which adipose tissue, preadipocytes, and adipocytes can be generated for research purposes, and methods for identifying cell populations that can proliferate and differentiate into adipocytes in vivo. The invention further provides a means for the in vivo derivation of “designer” or “customized” adipose tissue, preadipocytes, and adipocytes. Also provided are methods for identifying agents that affect adipocytes and adipose tissue, as well as the agents themselves. In particular, the present invention allows for creation of tissues and cells that can be used to screen for agents useful for treating human disorders associated with adipose tissue, including obesity, metabolic syndrome, and diabetes.

### FIELD OF THE INVENTION

The present invention relates to the field of medicine, specifically to methods and compositions useful for studying the biological properties of preadipocytes, adipocytes, and adipose tissue in vivo and in vitro, as well as for producing genetically-modified preadipocytes, adipocytes, and adipose tissue and for identifying cell populations capable of proliferating and differentiating into adipocytes in vivo.

### BACKGROUND OF THE INVENTION

Adipose tissue plays a significant role in energy metabolism and in human health. The ubiquitous presence of adipose tissue in mammals and in many non-mammals reflects its importance in energy storage, metabolism, as an endocrine organ, and in other areas that are only now being elucidated. Disorders associated with an excess of adipose tissue and a lack of it have been described. For example, type 2 diabetes mellitus occurs at a high rate not only in obese individuals, but also in patients with genetic disorders resulting in absence of adipose tissue, e.g., Berardinelli-Seip congenital lipodystrophy (BSCL) and in animal models such as the AZIP mouse (Moitra, et al., 1998, Genes Dev. 12(20):3168-81, incorporated herein by reference). The adipocytes themselves produce leptin, which regulates satiety and lipid metabolism. They also respond to insulin, which promotes lipid deposition into adipose tissue. Obesity, diabetes, cardiovascular disease, and other conditions associated with abnormal amounts and behavior of adipose tissue constitute a major international health problem. Improved understanding of the biology of human adipocytes and adipose tissue and the mechanisms by which they are generated and maintained will accelerate development of novel therapeutic approaches and agents to effectively treat these conditions.

Adipocytes arise from preadipocytes, which in turn are produced by a population of multipotent stem cells. Mature adipocytes are long-lived and are relatively resistant to apoptosis. The current understanding of the molecular basis of adipogenesis (the formation of adipocytes) has largely been developed based on studies of the 3T3 cell line and its many variants, including 3T3-L1, 3T3-F442A, and C3H-10T1/2 cells. These cells have several properties common to preadipocytes, including the ability to generate adipocyte-like cells in vitro and in vivo. When 10T1/2 cells are exposed in culture to bone morphogenic protein (BMP4) and then implanted into the subcutaneous space of immuno-incompetent (athymic) mice, they reportedly develop into a tissue that is similar to normal adipose tissue (Tang, et al., 2004, PNAS 101 (26):9607-9611, incorporated herein by reference). Injection of 3T3 cells into the subcutaneous space of an animal reportedly results in generation of adipose tissue or tissue resembling adipose tissue in that it includes regions comprised of large clusters of lipid-laden cells. These regions bear histologic similarity to primary adipose tissue. However, while the cells generated from these cell lines resemble adipocytes, there is evidence indicating that these cells are not representative of primary adult preadipocytes. For example, implantation of cultured preadipocytes into the subcutaneous space of animals has been reported to generate a transient adipose tissue that disappears after approximately two months (Patrick, et al., 2000, Semin. Surg. Oncol. 19(3):302-11, incorporated herein by reference). For example, TNF-α signalling events in human preadipocytes have been reported to differ substantially from those in 3T3-L1 adipocytes (Ryden, et al., 2002, J. Biol. Chem. 277(2): 1085-91, incorporated herein by reference). Differences are not completely unexpected, as 3T3 cells are an immortalized cell line originally derived from the embryo of albino Swiss mice, and 10T1/2 cells are derived from embryonic C3H mice. Furthermore, as early as 1980, Bjorntorp, et al., described age-specific and region-specific differences in rat preadipocytes (Bjorntorp, et al., 1980, J. Lipid Research 21:714-23, incorporated herein by reference). Clonally-derived cell lines such as 3T3 and 10T1/2 do not lend themselves to study of these differences. Similarly, disease, gender, and depot-related differences in preadipocyte and adipocyte biology are expected to be more effectively assessed using primary cells.

Subcutaneous and visceral adipose tissues have been reported to contain cell populations capable of in vitro differentiation into adipocytes (reviewed in Hausman, et al., 2001, Obesity Reviews 2(4): 239-54, incorporated herein by reference). Studies in which adult marrow-derived mesenchymal stem cells (MSC), cultured under conditions that induce adipogenic differentiation, showed certain characteristics of adipocytes, e.g., Oil Red O staining, have also been reported. These Oil Red O-positive cells generated in culture tend to be multilocular (and heterogeneously so) indicating that they are not mature (unilocular) adipocytes. Therefore, the use of cultured adult preadipocytes or stem cells is a limited means of generating mature adipocytes. This inhibits the use of such cells in studies of other aspects of mature adipocyte biology, such as apoptosis, and the development of adipose tissue as opposed to simply adipocytes.

Sekiya, et al., reported that while similar, there are distinct differences in gene expression seen in the adipogenic differentiation of 3T3 cells and marrow-derived cells (Sekiya, et al., 2004, J. Bone and Min. Res. 19(2):256-264, incorporated herein by reference). It is possible that these limitations and differences may be overcome at least partially by further optimizing culture conditions, for example, moving to three-dimensional cultures. Nonetheless, the process of in vitro differentiation remains cumbersome, expensive, labor-intensive and yields cells that are not fully representative of primary adipocytes.

Yuksel, et al., reported the in vivo generation of adipose tissue derived from host cells by implanting of a source of adipogenic growth stimulus, i.e., polymeric beads that slowly release insulin or insulin-like growth factor-1 (Yuksel, et al., 2000, Plastic and Reconstructive Surgery 105:1721-29, incorporated herein by reference). However, the duration of this study was only four weeks, which, in light of the studies by Patrick, et al., (Patrick, et al., 2000), is insufficient time to ascertain the stability of the tissue, particularly as the implanted beads continued to provide insulin throughout the four-week period. Since the resulting adipose tissue was derived from host cells, one would not be able to genetically modify the adipose tissue without genetically manipulating the host organism.

It is evident that different adipose tissue depots exhibit substantially different biological properties. In particular, excess visceral adipose tissue is associated with substantially increased risk for cardiovascular disease while excess peripheral (subcutaneous) adipose tissue is not. Further, in acquired lipodystrophies such as that frequently observed in patients receiving combination anti-HIV drug therapy, particularly those including protease inhibitors, certain adipose tissue depots have been observed to preferentially expand while others atrophy. Depot-related differences cannot be interrogated in a meaningful fashion using immortalized cells of fetal or embryonic origin such as 3T3 and C3H cells.

Investigators have examined primary cells having the capacity to differentiate into adipocytes and have found some differences in the biology of such cells in vitro. However, the difficulties described above in obtaining adipose tissue from primary cells in vivo are such that there has been little investigation of primary cells in obesity, cardiovascular disease, and adipogenesis.

Moitra, et al., 1998, have generated a mouse (commonly referred to as the A-ZIP mouse) that has essentially no white adipose tissue. This was achieved by introducing a dominant-negative protein, A-ZIP/F, which inhibits transcription factors critical for fat development, under the control of an adipose-specific promoter. More recently others have generated similar mice exhibiting inducible lipoatrophy by creating a system in which the same promoter is used to drive expression of an inducible gene that drives apoptosis. Other investigators have developed systems of intermediate phenotype and, more recently, Trujillo, et al., have described an inducible model of lipoatrophy (Trujillo, et al., 2005, Cell Cycle 4(9):1141-5, incorporated herein by reference). The severe form of lipoatrophy exhibited by A-ZIP animals results in insulin resistant diabetes and a metabolic syndrome similar to that observed in humans with congenital lipoatrophy and, ironically, in obese individuals. This syndrome can reportedly be resolved by transplantation of wild-type adipose tissue fragments but not by adipose tissue fragments from animals that do not express leptin. Implantation of adipose tissue fragments from a wild-type donor animal into insulin-resistant, hyperglycemic A-ZIP mice has been reported to result in return of insulin sensitivity and euglycemia. The use of lipoatrophic animals, to study cells derived from adipose-tissue and their capacity to become preadipocytes, adipocytes, and adipose tissue, has not been reported.

### SUMMARY OF THE INVENTION

The present invention overcomes the limitations of currently available methods for generating adipocytic cells in vitro and allows the generation of genetically modified mature adipocytes and adipose tissue without the need to derive transgenic animals or to co-implant growth factor delivery vehicles. This allows screening for drugs and other agents that modulate this process both in vivo (using tissues generated from native or genetically-modified cells) and in vitro (using native or genetically-modified preadipocytes or mature adipocytes. It further allows the identification and study of cell populations capable of forming adipocytes, preadipocytes, and adipose tissue in vivo.

Specifically, the invention relates to a method for generating adipocytes, comprising implanting cells capable of differentiating into adipocytes in a lipoatrophic host, and allowing said cells to form adipose tissue in said host. In embodiments, this method further comprises obtaining adipocytes from said adipose tissue. In other embodiments, the lipoatrophic host is immunotolerant. In yet other embodiments, the cells are human, and in others, the cells have been genetically modified.

The invention further relates to a composition comprising adipocytes obtained using the methods of the invention.

The invention includes a method of identifying a population of cells having the capacity to differentiate into mature adipocytes, or to proliferate and differentiate into mature adipocytes, comprising implanting the population of cells in a lipoatrophic host, allowing the population of cells to form tissue in the host, and detecting adipocyte generation and/or proliferation in the tissue formed. In embodiments, angiogenesis, arteriogenesis, or lymphangiogenesis are detected in the tissue. In other embodiments, the lipoatrophic host is immunotolerant. In embodiments, the population of cells is human. In further embodiments, the cells have been genetically modified.

The invention also relates to a method for generating soft tissue, comprising administering a compound comprising a cell population identified using the methods of the invention to an individual in need of soft tissue implantation or regeneration. In a specific embodiment, the invention relates to a method for generating soft tissue, for use in soft tissue implantation or regeneration, comprising administering a compound comprising a cell population that expresses CD73, does not express CD45 or CD31, and that expresses low levels of CD90 or no CD90.

The invention also includes a method of identifying an agent that modulates adipocyte generation or adipose tissue formation, comprising implanting cells capable of differentiating into adipocytes into a lipoatrophic host, allowing said cells to form adipose tissue in said host, comparing modulation of adipocyte generation or adipose tissue formation in the presence of an agent with modulation of adipocyte generation or adipose tissue formation in a control, and identifying an agent that substantially modulates adipocyte generation or adipose tissue formation relative to the control. In embodiments, the identified agent modulates the ability of adipose tissue to produce or respond to a biological response modifier. In specific embodiments, the biological response modifier can be a hormone or an adipokine. Further, it is contemplated that the identified agent modulates the angiogenic, lymphangiogenic, immunomodulatory, or arteriogenic activity, of the adipose tissue, adipocytes, or preadipocytes. Specifically, the identified agent can be used to treat an adipocyte-associated condition, e.g., obesity, diabetes, or obesity metabolic syndrome. Embodiments wherein the lipoatrophic host is immunotolerant, the cells are human, or the cells have been genetically modified, are also contemplated.

The invention provides a method of identifying an agent that that modulates a biological property of adipocytes, preadipocytes, or adipose tissue, comprising implanting a cell population capable of differentiating into adipocytes in a lipoatrophic host, allowing said cells to form adipose tissue in said host in the presence of an agent, measuring a biological property of adipocytes, preadipocytes, or adipose tissue, from the tissue formed in the presence of the agent and in a control, comparing the measurements made, in the presence of the test agent and in the control, and identifying the agent based on the comparison.

In embodiments, the identified agent modulates the ability of adipose tissue to produce or respond to a biological response modifier. In specific embodiments, the biological response modifier is a hormone or an adipokine. In other embodiments, the identified agent modulates the angiogenic, lymphangiogenic, immunomodulatory, or arteriogenic activity, of the adipose tissue, adipocytes, or preadipocytes. It is contemplated that the identified agent is used to treat an adipocyte-associated condition, e.g., obesity, diabetes, or obesity metabolic syndrome. In other embodiments, the lipoatrophic host is immunotolerant. In yet other embodiments, the cells are human, and in others, the cells have been genetically modified.

The invention additionally relates to a method of identifying an agent that modulates a toxic effect of a drug on adipocytes, preadipocytes, or adipose tissue, comprising implanting cells capable of differentiating into adipocytes in a lipoatrophic host, allowing said cells to form adipose tissue in said host, measuring the toxic effect of the drug on the adipocytes, preadipocytes, or adipose tissue, in the presence of a test agent and in a control, comparing the measurements made, in the presence of the test agent and in the control, and identifying the agent based on the comparison.

The invention further provides agents identified according to the methods of the invention, wherein the identified agent modulates the ability of adipose tissue to produce or respond to a biological response modifier, and in further embodiments wherein said biological response modifier is a hormone or an adipokine. In embodiments, the agent identified modulates the angiogenic, lymphangiogenic, immunomodulatory, or arteriogenic activity, of the adipose tissue, adipocytes, or preadipocytes. In specific embodiments, the agent is used to treat an adipocyte-associated condition, e.g., obesity, diabetes, or obesity metabolic syndrome. The identified agent can modulate the ability of adipose tissue to produce or respond to a biological response modifier, e.g., a hormone or an adipokine.

Some embodiments relate to methods for identifying an isolated population of adipose-derived regenerative cells capable of generating adipocytes or adipose tissue in a subject. The methods can include the steps of obtaining isolated adipose-derived regenerative cells from a subject; sorting the isolated adipose-derived regenerative cells into at least two different cell populations according to cell surface markers present on the cells; providing at least one of said at least two different cell populations to at least one host animal; and determining the presence, absence, quality, or amount of adipocytes or adipose tissue generated by the at least one of said two different cell populations in the host animal(s).

Other embodiments relate to methods for identifying a molecule that modulates a biological property of adipocytes or adipose tissue in a subject. In some embodiments, the methods can include the steps of obtaining isolated adipose-derived regenerative cells from a subject; providing the isolated adipose-derived regenerative cells to at least one host animal, such as a human, mouse, or other host animal; determining the presence, absence, quality, or amount of adipocytes or adipose tissue generated by the isolated adipose-derived regenerative cells in the host animal(s); providing a candidate molecule that modulates a biological property of adipocytes or adipose tissue to said host animal; and determining whether the candidate molecule modulates a biological property of adipocytes or adipose tissue in the host animal(s).

Yet other embodiments relate to methods for identifying a molecule that modulates the activity of a toxicant on adipocytes or adipose tissue in a subject. In some embodiments, the methods can include the steps of obtaining isolated adipose-derived regenerative cells from a subject; providing the isolated adipose-derived regenerative cells to at least one host animal; determining the presence, absence, quality, or amount of adipocytes or adipose tissue generated by the isolated adipose-derived regenerative cells in the host animal(s); providing the toxicant to the host animal(s); providing a candidate molecule that modulates the activity of a toxicant on adipocytes or adipose tissue to the host animal; and determining whether the candidate molecule modulates the activity of a toxicant on adipocytes or adipose tissue in the host animal(s). In some embodiments, the adipose-derived regenerative cells are sorted based on the presence or absence of cell surface markers on the adipose-derived regenerative cells prior to being provided to the host animal(s), and one or more of the subpopulations of sorted adipose-derived regenerative cells are provided to the host animal(s).

Other embodiments relate to methods of making an adipose-derived regenerative cell medicament. In some embodiments, the method can include the steps of isolated adipose-derived regenerative cells can be obtained from a subject; sorting the isolated adipose-derived regenerative cells into at least two different cell populations according to cell surface markers present on the cells; providing at least one of the two or more different sorted cell populations to at least one host animal; determining the presence, absence, quality, or amount of adipocytes or adipose tissue generated by the at least one of said two different cell populations provided to the host animal(s); and incorporating a cell population that is determined to generate adipocytes or adipose tissue in step (d) into a medicament.

Still other embodiments relate to methods of adipose-derived regenerative cell transplantation. In some embodiments, the transplantation methods can include the steps of obtaining isolated adipose-derived regenerative cells from a subject; sorting the isolated adipose-derived regenerative cells into at least two different cell populations according to cell surface markers present on the cells; providing at least one of said at least two different cell populations to at least one host animal; determining the presence, absence, quality, or amount of adipocytes or adipose tissue generated by the at least one of said two different cell populations in said the host animal(s); incorporating a cell population that is determined to generate adipocytes or adipose tissue into a medicament; and providing said medicament to a patient that is identified as one in need of adipose-derived regenerative cell transplantation.

In the embodiments described herein, the host animal(s) or subject(s) or both can be immunotolerant, syngenic, or lipoatropic, or any combination thereof.

In some embodiments of the methods provided herein, the presence, absence, quality, or amount of adipocytes or adipose tissue generated by at least two different cell populations sorted according to cell surface markers can be compared in either the same or different host animals.

In some embodiments of the methods provided herein, in a first model, the presence, absence, quality, or amount of adipocytes or adipose tissue generated by at least one of the at least two different cell populations sorted according to cell surface markers can be compared to a second model, wherein the presence or absence of adipocytes or adipose tissue generated by the isolated adipose-regenerative cells prior to cell sorting in either the same or different host animals are determined.

In some embodiments of the methods provided herein, the isolated adipose-derived regenerative cells can be sorted into at least two different cell populations according to cell surface markers present on said cells and at least one of said at least two different sorted cell populations are provided to at least one host animal to which the candidate molecule or the candidate molecule and toxicant are provided.

In some embodiments of the methods provided herein, the isolated adipose-derived regenerative cells can be sorted into at least two different cell populations according to cell surface markers present on the cells. At least two different sorted cell populations can be provided to at least one host animal to which candidate molecules (e.g., candidate agents that modify adipocyte, adipose tissue, or preadipocyte biological functions) or the candidate molecules in addition to a toxicant are provided. Optionally, the modulation of the biological property(s) of adipocytes or adipose tissue or the modulation of the activity(s) of the toxicant at the sites of introduction of the at least two different sorted cell populations are compared.

In some embodiments of the methods described herein, in a first model, the isolated adipose-derived regenerative cells can be sorted into at least two different cell populations according to cell surface markers present on said cells, and at least one of the at least two different sorted cell populations can be provided to at least one host animal to which the candidate molecule or the candidate molecule and toxicant are provided. In a second model, a portion of the isolated adipose-derived regenerative cells are provided to either the same or a different host animal to which the candidate molecule or the candidate molecule and toxicant are provided. Optionally, the modulation of the biological property of adipocytes or adipose tissue or the modulation of the activity of the toxicant in the two models can be compared.

In some embodiments, the isolated adipose-derived regenerative cells can be from a human. In some embodiments, the host animal can be a human, and in some embodiments, the host animal can be a mouse. In some embodiments, the subject from which the isolated adipose-derived regenerative cells are obtained and host animal, which receives said isolated adipose-derived regenerative cells, are the same species. For example, in some embodiments, the subject from which the isolated adipose-derived regenerative cells are obtained and host animal, which receives said isolated adipose-derived regenerative cells are the same individual.

In some embodiments, the isolated adipose-derived regenerative cells and/or a sorted cell population can be genetically modified prior to providing said the isolated adipose-derived regenerative cells and/or a sorted cell population to said host animal(s). For example, in some embodiments, the isolated adipose-derived regenerative cells and/or sorted cell population can genetically modified with a marker gene such as, GFP, luciferase, or B-gal.

Preferably, the isolated adipose-derived regenerative cells and/or a sorted cell population are isolated while maintaining a closed/sterile fluid pathway.

In some embodiments, the sorting step utilizes flow cytometry. For example, in some embodiments, the sorting step can be based on analysis of at least two cell surface markers, at least three cell surface markers, at least four cell surface markers, at least five cell surface markers, or at least six cell surface markers, or more.

In some embodiments, the at least two different cell populations can be provided to different host animals of the same species, whereas in other embodiments, the at least two different cell populations are provided to different host animals of different species.

In some embodiments of the methods described herein, the presence or absence of adipocytes or adipose tissue in the host animal(s) can be determined by measuring the appearance, size, morphology, or a biochemical marker of the adipocytes or adipose tissue. In some embodiments, the presence, absence, quality, or amount of adipocytes or adipose tissue in the host animal(s) can be determined by histology, staining, non-invasive detection of biological markers in the host animal(s), e.g., detection without sacrificing the animal, and the like. For example, in some embodiments, the determination of the presence, absence, quality, or amount of adipocytes or adipose tissue in said the host animal(s) can be determined by detection of a GFP without sacrificing the host animal(s).

In some embodiments, the methods further provide a step of determining the presence, absence, quality, or amount of angiogenesis, arteriogenesis, or lymphangiogenesis in said host animal.

In some embodiments, at least one of the two different cell populations that are provided to at least one host animal can expresses CD73, s not express CD45 or CD31, and expresses low levels of or no CD90.

In some embodiments, the modulation of activity is an up-regulation of activity, whereas in other embodiments, the modulation of activity is a down-regulation of activity. In some embodiments, the modulation can be up-regulation of one activity and down-regulation of another activity.

Exemplary candidate molecules useful in the methods described herein can hormones, adipokines, angiogenic modulating molecules, lymphangiogenic modulating molecules, immunomodulatory molecules, arteriogenic modulatory molecules, and the like.

In some embodiments of the transplantation methods described herein, the patient that is identified as one in need of adipose-derived regenerative cell transplantation can be a patient in need or that desires soft tissue implantation or regeneration. In some embodiments, the patient that is identified as one in need of adipose-derived regenerative cell transplantation is a patient with obesity, obesity metabolic syndrome, or diabetes. In some embodiments, the patient that is identified as one in need of adipose-derived regenerative cell transplantation is a patient with a cardiovascular disorder or peripheral vascular disease. In some embodiments, the method of claim 35, wherein the cell population provided to said patient expresses CD73, does not express CD45 or CD31, and that expresses low levels of CD90 or no CD90.

Other embodiments disclosed herein relate to the use of a medicament made in accordance with the methods described herein to treat a patient with diabetes, obesity, obesity, metabolic syndrome, a cardiovascular disease, or a peripheral vascular disease or a patient that desires soft tissue implantation, such as breast augmentation, or bone or disc replacement.

### BRIEF DESCRIPTION OF THE DRAWINGS

FIG. 1 Implant (highlighted within the black circle) derived from Matrigel™ supplemented with fresh (uncultured) adipose tissue-derived cells (see Example I). The picture was taken seven weeks after implantation. 1A. The implant in situ on the abdomen of the recipient mouse. A portion of a standard 2 cc syringe is shown for scale. 1B. The implant following dissection.

FIG. 2 Oil Red O Staining of implant. A histologic section of the implant shown in FIG. 1 was stained with Oil Red O to highlight cells having accumulated lipid (adipocytes). The Oil Red O staining (at 10× original objective) is seen as an even medium gray color and is indicated by arrows.

FIG. 3 Comparison of implants generated with Matrigel alone or Matrigel supplemented with adipose tissue-derived cells. 3A. An implant generated without cell supplementation showing transparency of the implant. 3B. A side-by-side comparison of Matrigel implants generated with and without cells.

FIG. 4 Histologic evaluation of an implant generated from matrigel supplemented with adipose tissue-derived cells. 4A. The implant, harvested at 12 weeks, shows Oil red O staining with 4× objective in the original. 4B. Shown at 20× original objective. 4C. Hematoxylin and eosin staining at 10× original objective.

FIG. 5 Oil Red O staining of an implant generated from collagen gel supplemented with adipose tissue-derived cells.

FIG. 6 6A. Hematoxylin and eosin staining of a region of tissue containing both adipocytes (clear, bubble-like structures on right) and non-adipocytes (nucleated cells in the fibrotic area to the left of the adipocytes) 6B. Fluorescence micrograph of the same region of the graft shown in 6A demonstrating that fluorescence is only visible within the region containing adipocytes. 6C. Higher magnification (40× original objective) of a different region of adipose tissue showing a cluster of fluorescent adipocytes.

FIG. 7 Histologic evaluation of an implant generated from collagen gel supplemented with cultured adipose tissue-derived cells. 7A. The implant, harvested at 12 weeks, shows hematoxylin and eosin staining. 7B. The same implant stained with Oil red O.

FIG. 8 Further histologic evaluation of an implant generated from collagen gel and cultured adipose tissue-derived cells. Adipocytes are marked with arrows, and regions of fibrosis containing non-adipocytes are marked with bars. 8A. Fluorescence microscopic evaluation of the implant. 8B. Hematoxylin and eosin staining of the same region demonstrating the distribution of adipocytes within this region.

FIG. 9 Histologic evaluation of an implant generated from Matrigel and cultured adipose tissue-derived cells. 9A. The implant, harvested at 12 weeks, shows hematoxylin and eosin staining. 9B. Oil Red O staining of the implant.

FIG. 10. CD45⁻/Sca-1⁻ Graft Histology. The tissue arising after implantation with the CD45⁻/Sca-1⁻ cell population was removed at 9 weeks and stained with Oil Red and Hematoxylin/Eosin. The dark areas indicate Oil Red O staining. A. In the graft from an animal (designated number 46, graft A), a few ORO-stained cells, mostly on the periphery of the graft, were observed. The H & E nuclear staining showed that the graft was largely acellular. B. In the other graft (number 46, graft B), many nucleated cells in the graft and loosely connected ORO-stained cells were observed. C. An area of graft 46B under increased magnification. D. H & E staining of the graft 46B. E. Image of ORO-stained tissue from animal 94, graft A. F. ORO-stained tissue from animal 94, graft B. The grafts shown in E. and F. had scattered, loosely associated, ORO-stained cells with little or no clustering.

FIG. 11. CD45⁻/Sca-1⁺/CD90⁻ Graft Histology. The tissue arising after implantation with the CD45⁻/Sca-1⁺/CD90⁻ cell population was removed at 9 weeks and stained with ORO and H & E. A. The image from an animal (36, graft A) showing a small graft with tight clusters of Oil Red O stained cells. B. The cell cluster seen at the upper right in A. under increased magnification. C. An image from another animal (41, graft A) showed many stained cells in clusters of 10 to 30. D. H & E staining of the graft 41A. E. An area of graft 41A under increased magnification. F. In the second ORO-stained graft from the same animal (number 41, graft B), many stained cells were observed in clusters. G. Image of ORO-stained tissue from animal 41, graft B. H. H & E staining of graft 41B.

FIG. 12. CD45⁻/Sca-1⁺/CD90⁺ Graft Histology. The tissue arising after implantation with the CD45⁻/Sca-1⁺/CD90⁺ cell population was removed at 5.4 weeks, after the animal (92) died prematurely, and stained with Oil Red O, and Hematoxylin/Eosin. A. Image of stained tissue animal (92, left graft, or 92L) showed scattered cells, some present in clusters. B. Another ORO staining image from 92L. C. H & E staining of graft 92L. D. ORO-stained tissue from animal 92, right graft (92R). E. ORO-stained tissue of graft 92R under increased magnification.

FIG. 13. CD45⁻/Sca-1⁺/CD31⁻/CD90^{low}/CD73⁺ Fluorescence Profile and Gating Strategy. A. Forward Scatter versus Side Scatter plot of cells. B. Plot of Sca-1 and CD45 expression of cells. C. Plot of Sca-1 and CD31 expression of CD45-negative/Sca-1-positive cells. D. Plot of CD90 and CD73 expression of CD45⁻/Sca-1⁺/CD31⁻ cells. D shows the two cell populations; on the lower right of the plot is the predominant population that is CD90⁺ and CD73⁻. To the left and above this population is the CD90^{low}/CD73⁺ population. E. Plot of CD90 fluorescence intensity of CD45⁻/Sca-1⁺/CD31⁻/CD73⁺ cells (light gray line in center). The isotype control for CD90 with this population (thin black line) and the CD90 expression of CD45⁻/Sca-1⁺/CD31⁻/CD90⁺/CD73⁻ cells (dark gray line on right) are shown for comparison.

FIG. 14. CD45⁻/Sca-1⁺/CD31⁻/CD90^{low}/CD73⁺ Graft Histology. The tissue arising after implantation with the CD45⁻/Sca-1⁺/CD31⁻/CD90⁻/CD73⁺ cell population was removed at 9 weeks. A. Staining with H & E. B. Staining with ORO.

### DETAILED DESCRIPTION OF THE INVENTION

The present invention relates to the discovery that freshly-extracted adipose tissue-derived cells and cultured adipose tissue-derived cells generate adipose tissue in vivo when implanted in lipoatrophic animals. The preadipocytes and adipocytes in the generated tissue carry the genotype of the donor cells. De novo generation of adipose tissue from human cells can be achieved through the use of a lipoatrophic animal that is also immunodeficient or immunotolerant. Further, implantation of genetically modified donor cells allows the generation of genetically modified mature adipocytes in the lipoatrophic host without the need to produce a new transgenic animal. In vivo tissues, as well as preadipocytes and mature adipocytes extracted from these tissues, can be used to study adipogenesis and to screen for drugs and therapies for treating conditions related to adipose tissue. For example, genetically modified tissues and cells can be used to screen agents for treating obesity.

The invention also relates to the identification of cell populations having the capacity to differentiate into adipocytes or proliferate and differentiate into adipocytes. These cell populations can be used for generating adipocytes, preadipocytes, and adipose tissue, and for identifying agents that affect adipocyte biology and have potential therapeutic use.

The evaluation of in vivo adipogenesis from human cells derived from individuals having normal body mass, obese individuals, individuals with acquired lipoatrophy such as HIV-associated lipoatrophy, inherited lipoatrophy, metabolic syndrome, type 1 or type 2 diabetes, systemic or local inflammation, and other conditions in which the biology of adipose tissue is or might be associated with a disorder are contemplated. The invention further relates to the study of cells derived from different adipose tissue depots (for example, subcutaneous or visceral) and the effects of implantation site (for example, subcutaneous, intramuscular, or intraperitoneal) on the biology of the implanted cells. In addition, implantation of genetically-modified human cells, from normal individuals and those with potential adipose-related disorders such as those listed above, is contemplated.

Citation of documents herein is not intended as an admission that any of the documents cited herein is pertinent prior art, or an admission that the cited documents are considered material to the patentability of the claims of the present application. All statements as to the date or representations as to the contents of these documents are based on the information available to the applicant and do not constitute any admission as to the correctness of the dates or contents of these documents.

### DEFINITIONS

In describing the present invention, the following terms will be employed, and are intended to be defined as indicated below. Unless otherwise indicated, all terms used herein have the same ordinary meaning as they would to one skilled in the art of the present invention.

As used herein, the term “adipocyte” refers to a cell that is specialized to synthesize and store fat. This term includes adipocytes with the properties representative of those present within white fat, yellow fat, and brown fat.

As used herein, the term “adipose tissue” refers to a tissue that contains adipocytes that may or may not be accompanied by stromal cells, blood vessels, lymph nodes, tissue macrophages, and other cells and structures. The term includes tissue that is commonly referred to in the art as white adipose tissue (or white fat), to brown adipose tissue (or brown fat), and to yellow adipose tissue (or yellow fat). Adipose tissue is normally found in multiple sites within the body including, but not limited to subcutaneous adipose, visceral adipose, omental adipose, perirenal adipose, scapular adipose, inguinal adipose, adipose surrounding lymph nodes, medullary adipose, bone marrow adipose, pericardial adipose, retro-orbital adipose, and infrapatellar adipose. In the context of the present invention the term “adipose tissue” also refers to tissue that contains adipocytes or preadipocytes, said adipocytes and/or preadipocytes being derived from implantation of donor cells capable of differentiating into preadipocytes and/or adipocytes. The term further includes tissue that does not yet contain adipocytes but which is a precursor or anlage of such tissue.

As used herein, the term “preadipocyte” refers to a cell capable of differentiating into an adipocyte. In particular, a preadipocyte contains little or no stored fat.

As used herein, the term “adipose tissue derived cell,” or “ADC,” refers to a heterogeneous population of cells derived as a result of the disaggregation of adipose tissue. In one embodiment the ADC population is largely depleted of adipocytes by exploiting the naturally low buoyant density of lipid-laden cells whereby such cells will float in commonly-used media while cells with little or no stored lipid will exhibit negative buoyancy and will sediment.

As used herein, the term “stem cell” refers to a cell with the ability to proliferate and to differentiate towards cells of more than one specialized cell type. For example, a cell that is capable of proliferating and of differentiating into cells with characteristics of adipocytes and into cells of the bone and/or of muscle fulfills this definition.

As used herein, the term “adipose derived stem cell,” or “ADSC,” refers to a cell derived from adipose tissue that is capable of proliferating and of differentiating towards cells of more than one specialized cell type.

As used herein, the term “adipogenesis” is a collective term that refers to the processes by which adipocytes are formed. The term applies both to the entire process by which an undifferentiated cell differentiates into an adipocyte and to steps within this process. For example, the term adipogenesis can apply to the maturation of a preadipocyte into an adipocyte, to the process by which precursors of preadipocytes (for example, stem cells) differentiate into preadipocytes, to combinations of such processes, and to subsets of the process by which a stem cell differentiates into an adipocyte.

As used herein, the terms “adipocyte biology” and “biological properties of adipocytes” refer to processes and properties directly pertaining to adipocytes, including processes involving and responses to compounds (e.g., biological response modifiers or drugs) participating in the formation (differentiation or proliferation), growth, metabolism, or death (programmed or otherwise) of adipocytes, preadipocytes, or any cellular intermediates in any of the listed processes and properties. Therefore, the terms refer to, e.g., the effect on these cells of agents that induce apoptosis, agents that alter gene expression, and agents that modulate the expression of genes associated with the synthesis and storage of fat. For example, the terms refer to the cells' responses to drugs affecting the synthesis and release of adipokines, e.g., adiponectin and leptin, and the cells' responses to insulin. Biological response modifiers, therefore, can modulate gene expression, e.g., cytokine or adipokine expression, or functional capabilities of the cells, such as response to insulin, or other factors, apoptosis, angiogenesis, arteriogenesis, etc., that can be measured or assessed using techniques known to those skilled in the art.

As used herein, the terms “adipose tissue biology” or “biological properties of adipose tissue” refer to processes and properties pertaining to the tissue that contains adipocytes (adipose tissue), including processes involving cellular elements of the tissue and also processes involving and responses of the tissue to compounds present within adipose tissue or forming adipose tissue. Cellular elements include, but are not limited to, adipocytes, cells that are not yet mature adipocytes but which are on the pathway to becoming adipocytes, cells that comprise structures within adipose tissue, including blood and lymph vessels and nodes (for example, blood vessel endothelial cells, lymphatic endothelial cells, pericytes, vascular smooth muscle cells, and lymph vessel smooth muscle cells), adipose tissue-resident macrophages, and other cells resident within adipose tissue. The terms refer to, e.g., the effect on adipose tissue of agents that induce apoptosis of cellular elements of adipose tissue, agents that alter expression of transcriptionally-regulated genes, and agents that modulate the expression of genes associated with the synthesis and storage of fat. Adipose tissue biology includes the growth of blood vessels and other structures in the tissue, the expression of proinflammatory cytokines, the synthesis and release of adipokines, e.g., adiponectin and leptin, and the responsiveness of the tissue to insulin. Therefore, the terms also refer to, e.g., the effect on adipose tissue of agents that interfere with the formation of blood vessels in growing adipose tissue, agents that modulate the expression of inflammatory mediators by adipose tissue-resident macrophages, and agents that modulate gene expression by adipose tissue-resident pericytes.

As used herein, the term “modulated” is intended to mean either upregulated or downregulated. For example, certain agents that modulate adipose tissue formation might increase or decrease the rate or extent of this process. The term is also meant to include maintenance of certain levels or rates in a potentially fluctuating parameter of interest, as maintenance can require modulation, e.g., in the form of alternating upregulation and downregulation.

### MODES OF CARRYING OUT THE INVENTION

It is to be understood that this invention is not limited to particular formulations or process parameters, as these may, of course, vary. It is also to be understood that the terminology used herein is for the purpose of describing particular embodiments of the invention only, and is not intended to be limiting. Further, it is understood that a number of methods and materials similar or equivalent to those described herein can be used in the practice of the present invention.

### 1. Methods of Obtaining Cells

While the use of freshly extracted adipose tissue-derived cells and cultured adipose tissue-derived cells are contemplated in the methods of the invention, the use of cells from other sources having adipogenic potential is also contemplated for in vivo generation of mature adipocytes. These cells include, but are not limited to; marrow stromal cells (MSC; also referred to as mesenchymal stem cells), cells from the outer ear (Rim, et al., 2005, FASEB J. 19(9): 1205-7, incorporated herein by reference), skin and skeletal muscle (Young, et al., 2001, The Anatomical Record 264: 51-62, incorporated herein by reference), blood vessels (Tintut, et al., 2003, Circulation 108(20): 2505-10, incorporated herein by reference), cartilage (de la Fuente, et al., 2004, Exp. Cell Res. 297(2): 313-28, incorporated herein by reference), and embryonic stem cells (Dani, et al., 1997, J. Cell Sci. 110(Pt 11): 1279-85, incorporated herein by reference). Cells from these and other sources may also be used for in vivo generation of mature adipocytes and can be obtained using methods known and described in the art. Cells with adipogenic potential from any source are contemplated for use in the present invention. As with adipose-derived cells, culture of non-adipose-derived cells with adipogenic potential and/or differentiation of such cells towards adipogenesis prior to implantation is contemplated for use in the methods of the present invention.

Adipose tissue is normally found in multiple sites within the body including, but not limited to subcutaneous adipose, visceral adipose, omental adipose, perirenal adipose, scapular adipose, inguinal adipose, adipose surrounding lymph nodes, medullary adipose, bone marrow adipose, pericardial adipose, retro-orbital adipose, and infrapatellar adipose.

### A. Adipose Tissue Derived Cells

In practicing the methods disclosed herein, the cells that are used to generate an adipose tissue-containing graft may be obtained from adipose tissue. Adipose tissue can be obtained by any method known to a person of ordinary skill in the art. For example, adipose tissue may be removed from a patient by suction-assisted lipoplasty, ultrasound-assisted lipoplasty, or excisional lipectomy. In addition, the procedures may include a combination of such procedures, such as a combination of excisional lipectomy and suction-assisted lipoplasty. Tissue may be obtained while the donor is living or dead, provided that the adipogenic cells remain viable. The tissue extraction should be performed in a sterile or aseptic manner to minimize contamination. Suction-assisted lipoplasty may be desirable to remove the adipose tissue from a human patient as it provides a minimally invasive method of collecting tissue with minimal potential for cell damage that may be associated with other techniques, such as ultrasound-assisted lipoplasty.

Means for obtaining adipogenic cells from adipose tissue have been described in the art. Most methods apply enzymatic digestion of washed adipose tissue fragments followed by centrifugation to separate buoyant adipocytes and debris from the non-buoyant cell fraction.

Adipose tissue processing can be performed by methods described in the literature and known to those of skill in the art, e.g., in U.S. application Ser. No. 10/316,127 (U.S. Pub. No. 2003/0161816), entitled SYSTEMS AND METHODS FOR TREATING PATIENTS WITH PROCESSED LIPOASPIRATE CELLS, filed Dec. 9, 2002, and U.S. application Ser. No. 10/877,822 (U.S. Pub. No. 2005/0084961), entitled SYSTEMS AND METHODS FOR SEPARATING AND CONCENTRATING REGENERATIVE CELLS FROM TISSUE, filed Jun. 25, 2004. The contents of both publications are expressly incorporated herein by reference. Preferably, the adipose tissue is processed in a stand-alone adipose tissue processing unit that isolates a population of adipose-derived regenerative cells while maintaining a closed, sterile fluid pathway. See, U.S. application Ser. No. 10/316,127 and U.S. application Ser. No. 10/877,822, above.

For suction-assisted lipoplastic procedures, adipose tissue can be collected by insertion of a cannula into or near an adipose tissue depot present in the patient followed by aspiration of the adipose into a suction device. In one embodiment, a small cannula may be coupled to a syringe, and the adipose tissue may be aspirated using manual force. Using a syringe or other similar device may be desirable to harvest relatively moderate amounts of adipose tissue (e.g., from 0.1 ml to several hundred milliliters of adipose tissue). Procedures employing these relatively small devices have the advantage that the procedures can be performed with only local anesthesia, as opposed to general anesthesia. Larger volumes of adipose tissue above this range (e.g., greater than several hundred milliliters) may require general anesthesia at the discretion of the donor and the person performing the collection procedure. When larger volumes of adipose tissue are desired to be removed, relatively larger cannulas and automated suction devices may be employed in the procedure.

Excisional lipectomy procedures include, and are not limited to, procedures in which adipose tissue-containing tissue (e.g., skin) is removed as an incidental part of the procedure; that is, where the primary purpose of the surgery is the removal of tissue (e.g., skin in bariatric or cosmetic surgery) and in which adipose tissue can be removed along with the tissue of primary interest (e.g., extraction of perirenal or omental adipose during abdominal surgery). Subcutaneous adipose tissue may also be extracted by excisional lipectomy in which the adipose tissue is excised from the subcutaneous space without concomitant removal of skin. Harvesting adipose tissue via excisional lipectomy of the inguinal fat depot is contemplated when using adipose tissue from mice.

The adipose tissue that is removed from a patient or animal can be collected into a device for further processing. Preferably, the adipose tissue is collected into a stand-alone adipose tissue processing unit that isolates a population of adipose-derived regenerative cells while maintaining a closed, sterile fluid pathway. See, U.S. application Ser. No. 10/316,127 and U.S. application Ser. No. 10/877,822, above.

The amount of tissue collected will be dependent on a number of variables including, but not limited to, the body mass index of the donor, the availability of accessible adipose tissue harvest sites, concomitant and pre-existing medications and conditions (such as anticoagulant therapy), and, in the case of research animals, the number of donors selected.

To obtain certain compositions in which the composition primarily contains one type of cell, any suitable method for separating the different cell types may be employed, such as the use of cell-specific antibodies that recognize and bind antigens present on either cell type. Similarly, subpopulations of certain cells can be isolated by use of negative selection approaches in which other cells are specifically removed. A fluorescently-labeled ligand can be used in FACS-based sorting of cells, or a ligand conjugated directly or indirectly to a solid substrate can be used to recover the cells of interest. Such methods are known in the art and are described herein and in the literature.

For most applications preparation of the active cell population will require depletion of the mature fat-laden adipocyte component of adipose tissue. This is typically achieved by a series of washing and disaggregation steps in which the tissue is first rinsed to reduce the presence of free lipids (released from ruptured adipocytes) and peripheral blood elements (released from blood vessels severed during tissue harvest), and then disaggregated to free intact adipocytes and other cell populations from the connective tissue matrix.

Rinsing is an optional, but preferred, step in which the tissue is mixed with solutions to wash off free lipid and single cell components, such as those components in blood, leaving behind intact adipose tissue fragments. In one embodiment, the adipose tissue that is removed from the donor is mixed with isotonic saline or other physiologic solution(s) (e.g., PLASMALYTE® physiolgical solution of Baxter Inc. or NORMOSOL® physiologic solution of Abbott Labs). Intact adipose tissue fragments can be separated from the free lipid and cells by any means known to persons of ordinary skill in the art including, but not limited to, filtration, decantation, sedimentation, or centrifugation. In embodiments of the invention, the adipose tissue is separated from non-adipose tissue by employing a filter disposed within a tissue collection container, as discussed herein. In other embodiments, the adipose tissue is separated from non-adipose tissue using a tissue collection container that utilizes decantation, sedimentation, and/or centrifugation techniques to separate the materials.

The intact tissue fragments are then disaggregated using any conventional techniques or methods, including mechanical force (mincing or shear forces), enzymatic digestion with single or combinatorial proteolytic enzymes, such as collagenase, trypsin, lipase, liberase H1, or members of the Blendzyme family as disclosed in U.S. Pat. No. 5,952,215, expressly incorporated herein by reference in its entirety, and pepsin, or a combination of mechanical and enzymatic methods. For example, the cellular component of the intact tissue fragments may be disaggregated by methods using collagenase-mediated dissociation of adipose tissue, similar to the methods for collecting microvascular endothelial cells in adipose tissue, as disclosed in U.S. Pat. No. 5,372,945, expressly incorporated herein by reference in its entirety. Additional methods using collagenase that may be used in practicing the invention are disclosed in U.S. Pat. No. 5,952,215, “Enzyme composition for tissue dissociation,” and by Williams, et al., 1995, incorporated herein by reference in its entirety. Similarly, a neutral protease may be used instead of collagenase, as disclosed in Twentyman, et al. (Twentyman, et al., 1980, Cancer Lett. 9(3):225-8), expressly incorporated herein by reference in its entirety. Furthermore, methods may employ a combination of enzymes, such as a combination of collagenase and trypsin or a combination of an enzyme, such as trypsin, and mechanical dissociation.

Adipose tissue-derived cells may then be obtained from the disaggregated tissue fragments by reducing the presence of mature adipocytes. Separation of the cells in the suspension may be achieved by buoyant density sedimentation, centrifugation, elutriation, filtration, differential adherence to and elution from solid phase moieties, antibody-mediated selection, differences in electrical charge; immunomagnetic beads, fluorescence activated cell sorting (FACS), or other means. Examples of these various techniques and devices for performing the techniques may be found in U.S. Pat. Nos. 6,277,060; 6,221,315; 6,043,066; 6,451,207; 5,641,622; and 6,251,295, all incorporated herein by reference in their entirety.

In one particular embodiment, the tissue is washed with sterile buffered isotonic saline and incubated with collagenase at a collagenase concentration, a temperature, and for a period of time sufficient to provide adequate disaggregation. Preferably, the tissue is washed in a stand-alone adipose tissue processing unit that processes adipose tissue to obtain a population of adipose-derived regenerative cells while maintaining a closed, sterile fluid pathway. See, U.S. application Ser. No. 10/316,127 and U.S. application Ser. No. 10/877,822, above.

In one embodiment, solutions contain collagenase at concentrations from about 10 μg/ml to about 50 μg/ml and are incubated at from about 30° C. to about 38° C. for from about 20 minutes to about 60 minutes. These parameters will vary according to the source of the collagenase enzyme, optimized by empirical studies, in order to confirm that the system is effective at extracting the desired cell populations in an appropriate time frame. A particular preferred concentration, time and temperature is 20 μg/ml collagenase (mixed with the neutral protease dispase; Blendzyme 1, Roche) and incubated for 45 minutes at about 37° C. An alternative preferred embodiment applies 0.5 units/mL collagenase (mixed with the neutral protease thermolysin; Blendzyme 3) and digests tissue for approximately 20 minutes.

Following disaggregation the active cell population can be washed/rinsed to remove additives and/or by-products of the disaggregation process (e.g., collagenase and newly-released free lipid). The active cell population can then be concentrated by centrifugation or other methods known to persons of ordinary skill in the art, as discussed above. These post-processing wash/concentration steps may be applied separately or simultaneously. Preferably, concentration steps are preformed in a stand-alone adipose tissue processing unit that isolates a population of adipose-derived regenerative cells while maintaining a closed, sterile fluid pathway. See, U.S. application Ser. No. 10/316,127 and U.S. application Ser. No. 10/877,822, above.

In addition to the foregoing, there are many post-wash methods that may be applied for further purifying the active cell population. These include both positive selection (selecting the target cells), negative selection (selective removal of unwanted cells), or combinations thereof.

Post-processing manipulation may also include cell culture or further cell purification. Mechanisms for performing these functions may be integrated within the described device or may be incorporated in separate devices.

By one approach, a population of adipose-derived regenerative cells capable of generating adipocytes or adipose tissue can be isolated and/or identified by obtaining isolated adipose-derived regenerative cells from a subject, and sorting the adipose-derived regenerative cells into at least two different cell populations according to cell surface markers present on the cells. The sorted cells can be provided to at least one host animal (e.g., a mouse or human host). The presence, absence, quality or amount of adipocytes or adipose tissue generated by the at least one of the sorted cell populations provided to the host(s) can be determined. See, e.g., Examples IV and V, below.

### B. Marrow-Derived Cells

In practicing the methods disclosed herein, the cells that are used to generate an adipose tissue-containing graft may be obtained from bone marrow, e.g., from a human. Bone marrow can be obtained by any method known to a person of ordinary skill in the art. For example, bone marrow may be removed from a patient by penetration and aspiration of the marrow cavity of the iliac crest, sternum, or other marrow cavity. Bone marrow may also be obtained from human donors undergoing bone resection or exposure of the marrow cavity for other purposes. Bone marrow from research animals or from cadaveric donors may be harvested by dissection of the femur or other bone, excision of the distal ends of the bone, and flushing the marrow cavity into a receptacle.

Bone marrow samples may optionally then be washed to remove contaminants such as bone spicules and medullary adipose, lysed to remove red blood cells, or subjected to differential density sedimentation or other approach that separates adipogenic cells (MSC) from some or all hematopoietic cells. Antibody-mediated positive or negative selection, cell adhesion, and cell culture, may also be applied in enrichment of adipogenic cells.

### C. Other Adipogenic Cells

In practicing the methods disclosed herein, the cells, e.g., human cells, capable of differentiating into adipocytes, which are administered to a patient may be obtained from tissues other than adipose tissue and bone marrow. For example, enzymatic digestion of skin, blood vessels, or skeletal muscle fragments has been shown to yield cell populations with adipogenic potential. Embryonic stem cells also possess adipogenic potential (Dani, et al., 1997) and may be generated by means that are known in the art and applied in the present invention.

Cell populations identified using the cell population identification methods according to the present invention can in turn be used to generate adipocytes and to identify agents that affect adipocyte biology. For example, a specific cell population identified based on its ability to differentiate into adipocytes, or to proliferate and differentiate into adipocytes, can be implanted in a lipoatrophic animal to generate adipocytes, generate soft tissue, or to identify agents that modulate adipocyte generation, proliferation of preadipocytes, or adipose tissue formation, agents that modulate the biological properties of adipocytes, preadipocytes, or adipose tissue, and agents that have a toxic effect on adipocytes, preadipocytes, or adipose tissue.

By one approach, a population of adipose-derived regenerative cells capable of generating adipocytes or adipose tissue can be isolated and/or identified by obtaining isolated adipose-derived regenerative cells from a subject, and sorting the adipose-derived regenerative cells into at least two different cell populations according to cell surface markers present on the cells. The sorted cells can be provided to at least one host animal (e.g., a mouse or human host). The presence, absence, quality or amount of adipocytes or adipose tissue generated by the at least one of the sorted cell populations provided to the host(s) can be determined. See, e.g., Examples IV and V, below.

Specifically, in some methods disclosed herein, following implantation, the implanted tissue can be excised and the cell/tissue mass can be measured. The cell/tissue mass of the implant can be compared with the cell/tissue mass of cells that were either not treated with a test compound (or treated with a placebo) prior to implantation. In some embodiments, the lipoatrophic animal, rather than the implanted cells are treated with a test compound (or placebo). The cell/tissue mass of similar cells implanted lipoatrophic animal that did not receive treatment with the test compound or agent, or received a placebo can be compared to the cell/tissue mass of the implant in the test animal which received treatment with a test compound. In some embodiments, the cell/tissue mass can be measured following excision. In other embodiments, the cell/tissue mass can be assessed using a detectable marker. For example, as discussed below, in some embodiments, the cell population can be genetically modified to express a detectable marker, such as luciferase, green fluorescent protein, or the like. The mass of tissue/cells derived from the implant can be assessed by invasive or non-invasive techniques known to those skilled in the art.

In some embodiments, a biopsy of the implanted tissue is performed. The biopsied cells can be assessed using routine histological techniques, such as the staining techniques described herein, to determine the presence/amount of adipocytes, or adipose tissue formation. The histology biopsied tissue can be compared with biopsied tissue of a control animal (i.e., a lipoatrophic animal that received an implant of untreated cells, or a lipoatrophic animal that did not receive treatment with the test compound or received a placebo).

In some embodiments, the cell population to be implanted can be contacted with a test compound or agent prior to implantation. In other embodiments, the animal is administered the test compound or agent (e.g., orally, intravenously, subcutaneously, or by any other method known to those skilled in the art) following implantation.

By one approach, a population of adipose-derived regenerative cells capable of generating adipocytes or adipose tissue can be isolated and/or identified by obtaining isolated adipose-derived regenerative cells from a subject, and sorting the adipose-derived regenerative cells into at least two different cell populations according to cell surface markers present on the cells. The sorted cells can be provided to at least one host animal (e.g., a mouse or human host). The presence, absence, quality or amount of adipocytes or adipose tissue generated by the at least one of the sorted cell populations provided to the host(s) can be determined. See, e.g., Examples IV and V, below.

### 2. Culturing Cells

Methods by which cells capable of participating in adipogenesis might be cultured are well known in the art. For example, Katz, et al. (U.S. Pat. No. 6,777,231), have described methods of culturing adipose tissue-derived stem cells. Similarly, Hamilton et al (U.S. Pat. No. 5,783,408, incorporated herein by reference) have described means for culturing preadipocytes for use in drug screening. Pittenger, et al. (U.S. Pat. No. 5,827,740, incorporated herein by reference) have described means by which mesenchymal stem cells may be cultured and induced to undergo adipogenesis. Dani, et al, (1997) have described means for culturing embryonic stem cells and inducing the cells to undergo adipogenesis. In general, these methods apply a basal cell culture medium to expand cell numbers followed by induction of adipogenesis by culturing cells in medium containing agents such as dexamethasone, activators of peroxisome proliferator-activated receptor gamma gene product, and insulin. Adipose-derived regenerative cells isolated as described herein (see, e.g. Examples I-V) can be cultured according to the teaching provided in this section.

### 3. Genetic Modification of Cells

Cells can be genetically modified, to express certain genes or to alter and even eliminate the expression of existing genes, using methods known to those of skill in the art. For example, by coupling the regulatory domain of the Bak gene to a reporter gene (as disclosed by Kiefer, et al., U.S. Pat. No. 6,436,639, incorporated herein by reference), transfecting cells capable of differentiating into adipocytes with this transgene, selecting cells that stably express the transgene, and implanting the selected cells in an animal as disclosed herein it is possible to generate adipocytes that express the reporter on induction of apoptosis. This system could be used in vivo with reporter genes (for example, luciferase, green fluorescent protein, β-galactosidase, or the like) that can be detected by non-invasive or minimally-invasive means or in vitro following extraction of the implant. For example, the DNA sequence comprising the active component of the Bak promoter can be inserted upstream of the gene encoding firefly luciferase gene using means that are well-known in the art. This construct can then be subcloned into an appropriate vector, permitting selection of stable ADSC transfectants. The use of a number of vectors to transfect ADSC has been reported in the literature (Morizono, et al., 2003, Hum. Gene Ther. 14(1):59-66) as has transduction with luciferase-containing vectors (Leo, et al., 2004, Spine 29(8): 838-44). Selected cells are then implanted into lipoatrophic mice as disclosed herein. Once adipose tissue has formed (as monitored by means such as those described herein) the tissue can be harvested to yield adipocytes that can be tested in vitro using screening techniques, including high throughput screening, for agents that modulate preadipocyte development, adipogenesis, adipose tissue formation, angiogenesis, arteriogenesis, and lymphangiogenesis, or combinations thereof. Methods for evaluating these processes in vitro are described in the literature and known by those of skill in the art. Alternatively, candidate agents can be administered in vivo and adipocyte apoptosis monitored by expression of the luciferase transgene.

This approach can be applied to essentially any adipocytic gene that is transcriptionally regulated. It can also be applied to evaluate the overall transcriptional activity of adipocytes by use of the promoter for an adipocytic “housekeeping gene.”

Other examples of genetic modifications that could be made to adipocytes generated according to the present invention include the introduction of mutant or polymorphic receptors and other molecules associated with adipogenesis, obesity or diabetes, for example, insulin receptor, Peroxisome Proliferator-Activated Receptor Gamma (PPARγ), aP2, leptin, and adiponectin). For example, the PPARγ gene has several polymorphisms in the normal population. Two exemplary PPARγ polymorphisms result in Pro115Gln and Pro12Ala.

Using the methods of the present invention it is possible to introduce genes encoding polymorphic proteins of interest into cells capable of undergoing adipogenesis and then generating adipose tissue and adipocytes carrying these forms in lipoatrophic animals. This provides a means for evaluating the biological consequences of such polymorphisms, for example, by comparing the effect of drugs that act on the PPARγ pathway on cells, tissue, or animals generated to have polymorphisms. Similarly, Klar et al., 2005, Eur. J. Hum. Genet. 13(8):928-34, have described a balanced chromosomal translocation in a family with profound, idiopathic obesity. The fusion gene created by the translocation is expressed in adipocytes. Using the present invention it is possible to generate adipocytes and adipose tissue that carry this fusion gene by transducing cells capable of undergoing adipogenesis with the gene and implanting them into lipoatrophic animals. These cells, tissues, and animals, could be used to screen for agents that impact the obesity associated with this translocation and to further understand adipogenesis.

### 4. Methods for Generating Adipocytes

### A. Lipoatrophic Hosts

In addition to the lipoatrophic mouse models currently available, the use of other lipoatrophic hosts in the methods of the present invention is contemplated. For example, rat, rabbit, and pig models of lipoatrophy could be used. It should also be understood that in some embodiments human are the host. In some embodiments, humans are the subjects from which the adipose-derived regenerative cells are obtained and the host to which the adipose-derived regenerative cells are provided.

i. AZIP Mouse

Aspects of the invention are based in part on the discovery that implantation of cells capable of differentiating into adipocytes (for example, adipocyte-depleted adipose tissue-derived cells, cultured adipose tissue-derived stromal cells, or adipose tissue-derived stem cells) into a lipoatrophic host results in the formation and long-term retention of tissue that contains adipocytes. The A-ZIP/F1 mouse (strain name FVB-Tg(AZIP/F)1 Vsn/J; Jackson Laboratories stock number 004100; described in Reitman, et al., 2000, Int. J. Obes. Relat. Metab. Disord. 24 (Suppl 4):S11-4), carries a dominant negative transgene that blocks the differentiation and maturation of preadipocytes thereby generating an animal that possesses essentially no white adipose tissue. Cross-breeding has been used to transfer the transgene from the FVB mouse background onto the C57BL/6J genetic background (Colombo, et al., 2003, J Biol Chem. 278(6):3992-9). These mice exhibit an absence of white adipose tissue, hyperglycemia, and insulin resistance. Transplant of wild type (but not leptin-deficient) adipose tissue fragments into A-ZIP mice reportedly results in restoration of insulin sensitivity and euglycemia.

ii. ATTAC Mouse

Unlike the A-ZIP mouse, the ATTAC mouse is an inducible lipoatrophic model. ATTAC is an acronym for “Adipose Tissue Targetted Activation of Caspase 8” (described in Pavjani, et al., 2005, Nature Medicine 11(7):797-803). The ATTAC mouse can be induced to express a chimeric molecule, part of which encodes a mutant form of the FKBP and part encoding the apoptosis activating gene caspase 8. Administration of an FK1012 analog (e.g., AP20187) leads to dimerization of the chimeric protein, resulting in apoptosis. In the ATTAC mouse the transgene is under the control of the promoter for the adipocyte/preadipocyte-specific gene aP2. Consequently adipose tissue in these animals is subject to inducible ablation creating a reversible lipoatrophy. Maintaining the drug prevents the animal from generating adipose tissue, creating a lipoatrophic phenotype.

ii. Lipoatrophic Immunodeficient Animals

Using strategies that are well-known in the art (such as cross breeding and the de novo generation of transgenic animals) it is possible to derive animals that are both lipoatrophic and immunodeficient. For example, on average one quarter of the offspring derived by breeding a heterozygous male A-ZIP mouse with a female mouse that is homozygous for the Prkdc^{scid} gene will carry both the A-ZIP phenotype and the Prkdc^{scid} gene. Back-crossing male progeny with this phenotype to females homozygous for the Prkdc^{scid} gene will yield progeny that are heterozygous for the A-ZIP gene and homozygous for the Prkdc^{scid} gene. Such animals will exhibit a lipoatrophic, immunodeficient phenotype. Consequently, it is possible to implant human adipogenic cells into such animals and, using the present invention, derive human adipocytes and adipose tissue that can be used for drug screening and other purposes as disclosed herein. Similar strategies using other lipoatrophic genotypes (for example, the ATTAC genotype) and other immunodeficiency genotypes (for example SCID beige animals) can produce a similar outcome though modifications in the strategy (for example additional generations of back-crossing) may be required to obtain the precise genotype of interest (for example, to fix the A-ZIP genotype onto the background of multiple mutations present in some immunodeficient animal models).

Host immunodeficiency in a lipoatrophic animal results in immunotolerance that permits the generation of adipocytes and adipose tissue from human individuals with particular diseases or characteristics of interest. For example, donor cells from obese persons or persons with a family history of or predisposition towards obesity or diabetes can be transplanted into the lipoatrophic animal. This strategy allows evaluation of the influence of donor age or the donor site (e.g., visceral, subcutaneous, or bone marrow) on adipogenesis. Combination of this approach with gene modification of the adipogenic cells provides a novel method of deriving human adipocytes, human adipose tissue, and developing human adipose tissue that can be used for research, drug screening, and other uses as disclosed herein.

### B. Transplantation of Cells

Delivery of cells, e.g. human cells, into the subcutaneous space, into muscle, into spaces between muscle groups, within the abdominal and thoracic cavities, or within the bone are contemplated. Methods for delivery are known in the art and described in the literature. Certain cells with the capacity to differentiate into adipocytes are capable of migrating to locations such as the medullary cavity of bone, to the spleen, or to other tissues (including perivascular tissue) following intravenous administration. Therefore, delivery via intravascular routes of administration is also contemplated. Cells can be delivered in suspension, in semi-solid carriers such as hydrogels, or in (or on) scaffolds such as woven and non-woven fiber-based scaffolds, sponges, and other highly porous structures. In embodiments such scaffolds can be engineered to include chemical or surface modifications that enhance attachment, proliferation, and/or differentiation and maturation of the cells into adipose tissue-like tissue.

Sponge-like scaffolds can be generated from thermoplastic substrates such as polyglycolide (PGA). Thermal compression of salt particles of defined size (generated by passing particles through sieves to generate a fixed size range) into preformed polymeric sheets followed by elution of the salt particles in an aqueous solvent generates scaffolds with high porosity, high pore interconnectivity, controllable pore size, and structural integrity. Similar scaffolds can be generated by a solvent-casting/freeze-drying/particulate leaching method and by other methods that are known in the art. These scaffolds can then be washed, sterilized, and seeded with cells (fresh cells, cultured cells, or cultured/predifferentiated cells) that can be implanted by injection or surgical insertion or other means. This approach provides a solid substrate to which the cells can attach and proliferate and/or differentiate. It further creates a space that is largely protected from forces generated by movement of skin against underlying structures, muscle against muscle, or in the intraperitoneal cavity. This is useful in the A-ZIP mouse model in which the underlying defect causes considerable hepatomegaly and a grossly enlarged abdomen. Similarly, a scaffold-like structure which simply maintains a protected space in which the implant can form in a hydrogel, scaffold, or other medium is also within the scope of the present invention. The chemical and physical properties of the polymer should be compatible with the biology of the cells and of the host.

Cell-seeded implants can be supplemented by loading the cells in a medium containing agents capable of promoting desired in vivo behavior, for example, Matrigel. Further, in vivo behavior can be modulated by coating the scaffold with Matrigel or other agents prior to implantation.

i. Injection

Cells, such as human cells, in suspension or loaded onto small scaffolds (for example beads or microbeads) can be implanted into host animals by injection. Implantation can be into the subcutaneous space, the peritoneal cavity, the medullary cavity of bone, or into other space (such as intramuscular or under the kidney capsule). Cells may be delivered on a bead-like or particulate scaffold using injection provided that a sufficiently large gauge needle is used such that the beads do not block the needle or that the application of injection force does not apply a degree of shear force to the beads or cells resulting in significant reduction of the integrity of the scaffold or viability of the cells. Cells may be injected in a simple aqueous solution such as physiologic saline or in an injectible hydrogel such as collagen or Matrigel. Many other injectible carrier materials are known in the art and have been described in the literature, e.g., peptide-based scaffolds, self-assembling materials, and synthetic polymers. In one embodiment cells are suspended in Matrigel and injected into the subcutaneous space of the dorsal flank of the lipoatrophic host at a concentration of 2 million cells per milliliter using a 16 G needle.

ii. Surgical Implantation

Cells, e.g., human cells may also be delivered to a host animal by surgical implantation. For example, cells may be seeded onto woven, non-woven, or molded scaffolds of defined porosity that are then placed within the desired site. For example, sponge-like scaffolds can be generated from thermoplastic substrates such as polylactide-coglycolide (PLGA). Thus, thermal compression of salt particles of defined size (generated by passing particles through sieves to generate a fixed size range) into preformed polymeric sheets followed by elution of the salt particles in an aqueous solvent generates scaffolds with high porosity, high pore interconnectivity, controllable pore size, and structural integrity. Similar scaffolds can be generated by a solvent-casting/freeze-drying/particulate leaching method. For example, a 3% solution of 85:15 polylactide-coglycolide in 1,4-dioxane may be produced and combined with salt particles (previously sieved to size range of 100-710 μm) at a polymer to salt ratio of 1:9. After most of the solvent evaporated to create a thick paste, the polymer-salt composite solution is frozen at −20° C. overnight. The frozen polymer-salt composite is then freeze dried for 8 hours to sublimate the frozen solvent crystals. This can yield a solid scaffold with 90% porosity that can be seeded with freshly prepared ADC or cultured ADSC. Seeded scaffold can be implanted directly or subjected to further culture in regular medium or in medium that induces adipogenesis. In one embodiment the host animal, e.g., mouse, is anesthetized with isofluorane and a 1 cm incision is made 1-2 cm to the right of the central line of the back. Blunt dissection is applied to open a small space to the side of the incision and the cell-seeded scaffold is inserted into this pocket. The incision is then closed by suturing. Additional implants may be placed along the other side of the back, in the ventral subcutaneous space (over the peritoneum) or in other convenient locations. Implantation within the visceral space (for example, within the peritoneal cavity or between muscles of the hindlimb) may also be applied. In the case of these deeper implants the skin, fascia, and muscle layers are closed according to standard surgical practice.

A combination of injection and surgical implantation may also be applied. For example, injection under the kidney capsule following surgical visualization of the injection site is contemplated.

### C. Adipocyte and Preadipocyte Identification and Assays

i. Protein Expression

Adipocytes can be identified, e.g., by their characteristic morphology, buoyancy, or expression of specific markers such as aP2, lipoprotein lipase, or leptin. Gene expression in preadipocytes and adipocytes has been described in the literature and is compared, e.g., by Urs, et al., 2004, J. Nutr. 134:762-770, which describes differential gene expression in relation to cellular function. Preadipocytes can also be identified by their ability to differentiate into adipocytes, as understood by those of skill in the art. The levels of the markers can be measured using immunological or molecular biological techniques known to those skilled in the art, such as ELISA, immunoblot, RNA amplification techniques, and the like.

ii. Histological Evaluation

Adipocyte differentiation can be further evaluated based on histological analysis. Methods for staining accumulated triglycerides with Oil Red O or Osmium Tetroxide are known to those of skill in the art. The characteristic lace-like morphology of adipose tissue as evident in staining of sections with hematoxylin and eosin may also be used to evaluate adipogenesis. Methods for quantitating Oil Red O-staining cells are known and described in the literature. By way of example, Ramirez-Zacarias, et al., 1992, Histochemistry 97(6):493-7 reports on a method of quantitating cell differentiation by measuring lipid accumulated in the cytoplasm of cultured 3T3 cells using Oil Red O stain and measuring the amount of extracted dye at 510 nm.

5. Identification of Cells Having the Capacity to Proliferate and Differentiate into Mature Adipocytes

The methods of the present invention can be used to identify cells that can proliferate and/or differentiate into mature adipocytes in vivo. Identification can be carried out, e.g., as described in Examples IV and V, wherein 1,000 or 10,000 cells from each of several sorted cell populations were implanted in a GFP-expressing lipoatrophic mouse. It is understood by those of skill in the art that an appropriate number of cells to be implanted can vary depending on the site of implantation, the size and species of animal, the number of cells available, and other factors that one of skill in the art can evaluate. Implanted cells are allowed to grow, and after a period of time, which can be varied as desired by the researcher, the graft can be analyzed.

Analysis or measurement of cell differentiation in the graft can be made using any method desired and available to one of skill in the art. For example, as described herein in the Examples, the graft can be removed and sections can be stained with reagents that indicate adipogenesis, e.g., Oil Red O, as described elsewhere herein and in the literature. The graft can further be analyzed for angiogenesis, arteriogenesis, and/or lymphangiogenesis using methods and markers well known to those of skill in the art and described in the literature. For example, vascular structures can be stained in vivo for endothelial and smooth muscle cell markers that include, but are not limited to, CD31, von Willebrand Factor VIII, smooth muscle actin, and smooth muscle myosin. Lymphatic structures can be stained for lymphatic endothelial cell markers that include, but are not limited to, FLT-4 (also referred to as VEGF receptor-3, or VEGFR-3), D2-40, the homeobox-containing gene Prox-1, podoplanin, and the CD44 homolog LYVE-1. Immunological techniques, e.g., ELISA, immunoblots, as well as gene expression techniques, e.g., RNA amplification, blotting, and the like, can be used to assess or measure cell differentiation.

The ability of donor cells to become blood or lymphatic endothelial cells, or other cell types, can thus be measured or assessed. Some level of blood and lymphatic vessel formation is expected to occur along with adipose tissue formation. Formation of these supportive tissues can be important in adipogenesis. For this and other reasons, it can be desirable to identify a cell population that has an effect on adipogenesis as well as angiogenesis, arteriogenesis, or lymphangiogenesis. Furthermore, the methods of the invention can allow the identification of agents that can modulate two or more of these processes, e.g., adipogenesis and angiogenesis.

Proliferation can be evaluated and/or measured based on the pattern of cell growth, for example, a tight clustering of adipocytes is indicative of proliferation. BrDU incorporation can be used to detect proliferating cells in situ, as can proliferating cell nuclear antigen (PCNA) IHC, Ki-67 IHC, and in situ hybridization for histone mRNA. These methods are described by, e.g., Hewitson, et al., 2006, Methods Mol. Biol. 326:219-26, and Muskhelishvili, et al., 2003, J. Histochem. & Cytochem. (51)12:1681-1688.

Differentiation and/or proliferation can be measured and/or compared in grafts generated using different cell populations. This provides additional information about cells that can be useful when evaluating their ability to produce healthy tissue for therapeutic use. Depending on the therapeutic or other use contemplated for the test cells, different assays known to those of skill in the art can be used to test the graft.

### 6. Identification of Modulating Agents

### A. Candidate Modulating Agents

The methods of the invention can be used to identify agents that modulate biological properties of adipocytes, preadipocytes, or adipose tissue. In these methods, a cell population capable of forming adipocytes, preadipocytes, or adipose tissue, is implanted in a lipoatrophic host. As described above, cell populations contemplated for implantation in practicing methods of screening agents that modulate biological properties of adipocytes, preadipocytes, or adipose tissue include populations identified based on their ability to differentiate into adipocytes, or to proliferate and differentiate into adipocytes, according to methods of the present invention.

By one approach, adipose-derived regenerative cells are isolated from a subject. The adipose-derived regenerative cells provided to at least one host animal, and the presence, absence, quality, or amount of adipose-derived generated by the regenerative cells provided to the host is determined. The host can be provided with a candidate molecule that modulates a biological property of adipocytes or adipose tissue, and it can be determined whether the candidate compound modulates a biological property of adipocytes or adipose tissue in the host, as described herein. In some embodiments, the adipose-derived regenerative cells are obtained as described in Examples IV and V herein.

Agents that might be applied in screening include agents that might, e.g., stimulate or slow the generation of adipocytes, preadipocytes, and adipose tissue, and agents that might be toxic to adipocytes, preadipocytes, and adipose tissue. Candidate agents include but are not limited to: small molecules, e.g., those that interact with G protein coupled receptors; peptides and polypeptides, for example, growth factors and growth factor receptor blockers or agonists (examples of these in other settings include etanercept, infliximab, and anakinra, reviewed in Symmons, et al., 2006, Lupus 15(3): 122-6); polynucleotides, for example, aptamers, small interfering RNA molecules or antisense oligonucleotides (reviewed in Tafech, et al., 2006, Curr. Med. Chem. 13(8): 863-81) or polynucleotides that include coding sequences for same or for other regulatory molecules (Boghossian, et al, 2005, Peptides 26(8):1512-9); and lipids or lipid-containing molecules, for example, prostaglandins and myristoylated peptides or polypeptides (Xie, et al., 2006, Chem. Pharm. Bull. (Tokyo) 54(1): 48-53). Contemplated targets for such molecules include, but are not limited to, PPAR-γ, beta-3 adrenergic receptor, hormone sensitive lipase, adiponectin, leptin, Interleukin 6, Interleukin 10, and molecules that play a role in the regulation of production of potential targets.

The invention contemplates the identification of agents that alter the biology of adipose as a multicellular tissue. It is apparent that adipose tissue formation involves cross-talk between different cellular elements; for example, preadipocytes, adipocytes, and developing vascular cells (Rupnick, et al., 2002, PNAS 99(16):10730-5; Hausman, et al., 2004, Journal of Animal Science 82:925-34). In embodiments of the invention, the identification of agents that interfere with or otherwise alter this cross-talk is contemplated. For example, it is possible to use gene transfer technology to impair the expression or function of certain molecules within ADSC. By using ADSC that have been genetically modified, it is possible to create adipose tissue and adipocytes that are more (or less) sensitive to certain physiologic or pharmacologic stimuli and, thereby, screen for agents that alter adipocyte biology in this different background. For example, stable transduction of ADSC with genes encoding small interfering RNA molecules, dominant-negative gene forms, or novel genes (for example, the chimeric gene used in generating the ATTAC transgenic mouse described by Pajvani, et al., 2005) can result in models for studying adipogenesis, preadipocytes, or mature adipocytes, in which the cells express a non-wildtype genetic background. Such models can be used to screen for agents that alter various aspects of adipocyte and/or adipose tissue biology.

In embodiments of the present invention, agents are identified that function through both direct and indirect regulation. For example, the agent applied in screening tests may directly upregulate the expression of a reporter gene, or the agent might directly change the expression or activity of other molecules such as receptors, signal transduction molecules, or molecules involved in regulation of the cell cycle or apoptosis and thereby indirectly modulate the expression of the reporter gene.

### B. Modulation of Adipocyte Generation

New adipocytes can be generated by a number of different mechanisms including the maturation of cells containing little or no intracellular lipid storage depots into mature adipocytes. This process can be measured by methods known and described in the art, e.g., counting Oil Red O-positive cells generated in the presence of a molecule that blocks cell division. Proliferation and maturation can also be measured using assays known in the art, for example by measuring incorporation of active DNA synthesis (e.g., tritiated thymidine), or by labeling an adipocyte-free cell population and detecting the subsequent appearance of labeled adipocytes in the population.

For example, adipose tissue-derived cells can be prepared and injected into the subcutaneous space of lipoatrophic mice as described herein in the examples. The animals can be treated with a candidate agent, and screened for adipogenesis. Adipogenesis can be measured or detected, e.g., by measuring the presence and levels of leptin or other products or markers of the presence of adipocytes or functional preadipocytes in the blood, using, e.g., magnetic resonance spectroscopy. Alternatively, the donor cells can be genetically modified to express a marker gene such as luciferase, green fluorescent protein, or the like, under the control of an adipocyte/preadipocyte-specific promoter, the expression of which indicates de novo adipogenesis.

Delivery of human cells, capable of differentiating into adipocytes, into a lipoatrophic animal incapable of mounting an effective immune response to the implanted material allows the generation in vivo of adipose tissue composed of human cells. This animal can be used to screen or test for agents that modulate human adipogenesis or adipo-toxicity in vivo.

It is also known that different adipose tissue depots confer different risks for cardiovascular disease and diabetes. The present invention contemplates the generation of adipose tissue using cells from different depots to evaluate: agents that differentially affect the development of adipose by different tissues; molecules that are differentially expressed by cells from different depots, or; molecules that can convert the phenotype of one depot (for example, visceral adipose) to that of another (for example, subcutaneous adipose).

Cell populations contemplated for implantation in practicing methods of identifying agents that modulate adipocyte differentiation include populations identified based on their ability to differentiate into adipocytes, or to proliferate and differentiate into adipocytes, according to methods of the present invention. By one approach, the cell population used for screening or identifying agents that modulate adipocyte differentiation is isolated by the methods taught in Examples IV and V, below. Briefly, adipose-derived regenerative cells are obtained from a subject. The adipose-derived regenerative cells sorted into at least two different cell populations according to cell surface markers present on the cells. At least one of the subpopulations of sorted cells can be used in the screening methods described herein. See, e.g., Examples IV and V, below.

### C. Modulation of Adipose Tissue Formation

Processes for generating or expanding the tissue containing adipocytes involve adipocyte generation (through differentiation and proliferation, i.e., “hyperplasty”) and/or an increase in the size of existing adipocytes (“hypertrophy”). In hypertrophy, adipose tissue mass increases as a result of increased lipid content within existing adipocytes. However, the ability of individual cells to expand in size and store additional lipid is limited. Once this limit is reached, additional lipid storage and increased adipose tissue mass is accommodated by generation of additional adipocytes (hyperplasty), which involves the formation of new adipocytes derived from populations of cells with the ability to differentiate into adipocytes (e.g., ADC or ADSC). These cells include adipocyte precursors (preadipocytes), adipogenic progenitors (Adipocytic-Colony-Forming Units; CFU-Ad), and multipotent stem cells. Hypertrophy can be evaluated by measuring the size of adipocytes or by quantitating changes in the number of adipocytes in a particular volume of tissue. Hyperplasty can be assessed, e.g., by determining the absolute number of adipocytes within a particular adipose tissue depot or implant or by methods described herein.

Adipose tissue formation occurs concomitantly with and is associated with the development of blood vessels that supply the growing tissue. It also occurs along with colonization by other cells and structures including lymph nodes, lymph vessels, and tissue macrophages, all contained within the tissue.

The methods of the invention contemplate the evaluation of agents that alter adipose tissue formation. For example, candidate agents can be tested in animals that have received implants as described herein and the animals monitored for the formation of new adipose tissue. Screening for new tissue formation can be performed by many different means including, but not limited to, harvest of implant tissues and histologic evaluation of the tissue as described herein, measurement and evaluation of blood glucose to determine if sufficient tissue has developed to engender a resolution of insulin resistance, blood testing and measurement of factors secreted by adipocytes (for example, leptin or adiponectin), magnetic resonance imaging or other non-invasive means of detecting and measuring adipose tissue, and use of genetically modified cells (for example, cells expressing the gene for luciferase, green fluorescent protein and the like) which would allow for non-invasive monitoring of engraftment by donor cells (Leo, et al., 2004). These means can be used to screen for agents that promote or inhibit adipose tissue formation. Detection of other cells and structures (e.g., lymph vessels, blood vessels, and macrophages) is performed in a similar manner using markers that are deemed specific for such elements (e.g., podoplanin, VE cadherin, and CD14).

Cell populations contemplated for implantation in practicing methods of identifying agents that modulate adipose tissue formation include populations identified based on their ability to differentiate into adipocytes, or to proliferate and differentiate into adipocytes, according to methods of the present invention. By one approach, the cell population used for screening or identifying agents that modulate adipose tissue formation is isolated by the methods taught in Examples IV and V, below. Briefly, adipose-derived regenerative cells are obtained from a subject. The adipose-derived regenerative cells sorted into at least two different cell populations according to cell surface markers present on the cells. At least one of the subpopulations of sorted cells can be used for the screening/identification methods described herein. See, e.g., Examples IV and V, below.

### D. Modulation of Biological Properties

The methods of the invention can be used to identify agents that affect the biological properties of adipose tissue, preadipocytes or adipocytes, as discussed herein. For example, the ability of adipose tissue, preadipocytes or adipocytes to produce or respond to biological response modifiers such as hormones, e.g., insulin, and adipokines, e.g., leptin. Using a reporter gene or other system that allows quantitation of leptin production, the methods of the invention can be used to screen for agents that alter the expression or production of leptin without the need for a specific bioassay for leptin. This can be performed in vivo or in vitro. Similarly, altered insulin sensitivity can be detected using the methods of the invention.

Cell populations (e.g., human cells) contemplated for implantation in practicing methods of identifying agents that modulate biological properties of adipocytes, preadipocytes, or adipose tissue, include populations identified based on their ability to differentiate into adipocytes, or to proliferate and differentiate into adipocytes, according to methods of the present invention. By one approach, the cell population used to identify agents that affect the biological properties of adipose tissue, preadipocytes, or adipocytes, is obtained by obtaining isolated adipose-derived regenerative cells from a subject, and sorting the adipose-derived regenerative cells into at least two different cell populations according to cell surface markers present on the cells. One or more of the subpopulations of sorted cells can be used for the screening methods described herein. See, e.g., Examples IV and V, below.

i. Modulation of Immunomodulatory Activity

One biological property of adipocytes and adipose tissue is their reported immunomodulatory activity, i.e., their influence on the immune system and inflammation (as described by, e.g., Trayhurn, et al., 2004, British J. Nutrition 92: 347-355). In particular, a number of studies have reported that obesity is associated with a low grade systemic inflammation and that different cells within adipose tissue secrete a number of biological response modifiers, including molecules that can modulate the immune system and inflammation. Trayhurn, et al., 2004, report agents involved in inflammation (Tumor Necrosis Factor-α, Interleukin 6 (IL-6), IL-10, IL-8, IL-1β, Transforming Growth Factor-β, Nerve Growth Factor) and in the acute phase response (plasminogen activator inhibitor-1, haptoglobin, serum amyloid A). Immune cells within adipose tissue also reportedly exhibit properties that appear to be distinct from immune cells circulating in the blood.

Using the methods of the present invention, agents that alter the expression of immunomodulatory and immune regulatory molecules by adipose tissue, preadipocytes, or mature adipocytes, can be identified. Agents that alter the responsiveness of adipose tissue, preadipocytes, or mature adipocytes to immunomodulatory molecules can also be identified.

For example, using the methods of the present invention, adipose tissue can be generated using ADSC carrying a reporter gene under the control of the IL-6 promoter. Adipocytes generated in vivo from these cells using the present invention can then be used in in vitro high throughput screening to define agents capable of modulating expression of IL-6.

Similarly, it is possible to use the present invention to generate a localized depot of adipose tissue, to deliver agents directly to this depot, and then evaluate and/or measure the effects of such agents on the ability of the adipose tissue and cells and structures within the adipose tissue to perform a particular biologic function (for example, expression of interleukin 6). In one embodiment this approach is used to evaluate depot-specific effects of candidate agents. For example, using the methods of the present invention it is possible to generate adipose tissue using cells derived from subcutaneous adipose tissue or from visceral adipose tissue; two depots with well-described, different, biological properties. The methods of the present invention enable adipose tissue to be generated in the subcutaneous space or within the peritoneal cavity. Thus, using the present invention it is possible to evaluate the effects of a particular agent on the biological properties of subcutaneous or visceral adipose in an animal having adipose that exhibits properties of just one of these depots and to detect agents that exhibit depot-selectivity.

The same general approach could be applied to in vivo screening to evaluate expression of biological response modifiers in the whole animal context. Similarly, it can be applied to agents that alter expression of ancillary molecules such as matrix metalloproteinases (including, for example the membrane-anchored metalloproteinase, MT1-MMP), adhesion molecules, receptors (including, for example, members of the integrin superfamily), signal transduction molecules (including, for example, members of the Jak/stat family), transcriptional regulators (including, for example, members of the CAAT/enhancer-binding protein (C/EBPs) and peroxisome proliferator-activated receptor (PPAR) families, and extracellular matrix molecules (including, for example, collagen and laminin).

i. Modulation of Adipocyte Death

The present invention contemplates methods useful for identifying agents that modulate apoptosis of adipocytes. For example, the present invention can be applied using cells that have been modified to carry a reporter gene (for example, the gene encoding firefly luciferase, green fluorescent protein, or the like) under the control of a promoter that is activated during apoptosis (for example the promoter for the Bak gene). Gene modification can be achieved by a number of means known in the art, for example, by use of a retroviral construct. Transduction of adipogenic cells using such means is well known in the art and is described, e.g., by Morizono, et al., 2003 and Dragoo, et al., 2003, J. Orth. Res. 21:622-629, incorporated herein by reference. Adipocytes generated, using the methods of the present invention, from genetically modified cells, would carry the transgene. Induction of apoptosis in the adipocytes would be associated with luciferase expression, allowing detection of apoptotic cells and screening for the agents that induce apoptosis.

### E. Modulation of Toxic Effects of Drugs

In developing any new drug or therapy it is important to evaluate the potential for side-effects such as toxicity to non-target. For example, it would be important that a new drug designed to increase bone strength in osteoporotic persons would not result in ablation of adipose tissue as this could lead to insulin resistance and type 2 diabetes. It is understood that a new treatment for almost any condition should not induce lipoatrophy or, at least, that such an effect would be only temporary.

The present invention permits the generation of efficient models in which the toxicity of agents towards adipose tissue can be evaluated. Further, given the importance of angiogenesis in adipose tissue development (Rupnick, et al., 2002), the present invention provides methods of screening for agents that mediate toxicity by inhibiting blood vessel formation. Candidate agents can be administered to the animals at any point after implanting cells, and toxicity monitored by means such as those described herein, for example, histologic evaluation of the implant, magnetic resonance imaging, use of reporter genes, and blood tests for adipose-related genes. For example, in some embodiments, adipose-derived regenerative cells are obtained from a subject, and provided to at least one host animal. The presence, absence, quality, or amount of adipocytes or adipose tissue generated by the isolated adipose-derived regenerative cells in the host animal is determined. The animal can be provided a toxicant, and a candidate compound/agent, and the modulation of the activity of the toxicant on the adipocytes or adipose tissue can be determined.

In other embodiments, adipose-tissue and adipocytes derived from adipose-derived regenerative cells as described in Examples I-II can be used to screen candidates that modulate the activity of toxicants, or biological properties of adipocytes or adipose tissue in vitro.

Cell populations contemplated for implantation in practicing methods of identifying agents that modulate the toxic effect of drugs on adipocytes, preadipocytes, and adipose tissue include populations identified based on their ability to differentiate into adipocytes, or to proliferate and differentiate into adipocytes, according to methods of the present invention.

### F. Modulation of Angiogenic, Lymphangiogenic, and Arteriogenic Activity of Adipose Tissue, Preadipocytes or Adipocytes

The processes of angiogenesis, arteriogenesis, and lymphangiogenesis play a key role in e.g., embryonic development, wound healing, and tissue regeneration. There is evidence that adipose tissue mass is affected by modulation of angiogenesis, and that there is a regulated relationship between adipose tissue and lymph nodes. Furthermore, cells from adipose tissue have been reported to be involved in wound healing (e.g., by El-Ghalbzouri, et al., 2004, Br. J. Dermatol. 150(3) 444-54). Agents that modulate angiogenesis, arteriogenesis, and lymphangiogenesis can be used to modulate the formation of adipose tissue, and to modulate the effect of adipose-derived cell populations on tissue regeneration and wound healing.

As previously discussed, adipose tissue and the cells present therein can modulate the formation and expansion of blood vessels and lymphatic vessels. This phenomenon is reportedly mediated, at least in part, by expression of pro-arteriogenic, angiogenic, and lymphangiogenic factors by adipose tissue, preadipocytes, and mature adipocytes. The methods of the present invention can be used to identify agents that alter the ability of adipose tissue, preadipocytes, or mature adipocytes to mediate these effects and to express pro-arteriogenic, angiogenic, and lymphangiogenic factors. These effects involve cross-talk between different cells within adipose tissue. Thus, using the methods of the present invention, agents that interfere with this cross-talk and thereby alter the angiogenic, arteriogenic, and/or lymphangiogenic properties of the tissue and/or cells can be identified. Alteration of pro-arteriogenic, angiogenic, and lymphangiogenic properties can be evaluated and/or measured, e.g., by monitoring altered expression of molecules which regulate or mediate such processes. Examples of such molecules are Placental Growth Factor, Hepatocyte Growth Factor, receptor molecules, secondary mediators such as matrix metalloproteinases that act on tissue remodeling, and inhibitors and activators thereof.

The methods of the present invention can be used to identify agents that modulate the angiogenic activity of adipocytes, preadipocytes, or adipose tissue. An identified agent can modulate the development of new blood vessels or the expansion of pre-existing blood vessels. For example, lipoatrophic A-ZIP mice may be cross-bred with animals that are transgenic for a marker gene, for example, FVB/N-Tg(TIE2-lacZ)182Sato/J mice (Jackson Laboratories). This strain is particularly useful as the A-ZIP mouse was originally created on the FVB mouse background and, consequently, the two mice are largely congenic. This cross-breeding creates a strain of mouse that is lipoatrophic and in which the lacZ transgene is expressed exclusively in endothelial cells. Therefore, adipose tissue-derived cells can be prepared from wild-type FVB mice and injected into the subcutaneous or intraperitoneal space of A-ZIP/Tie2lacZ lipoatrophic mice as described herein. The vasculature of the newly-formed adipose tissue formed thereby will include host-derived endothelial cells. Agents that modulate in vivo angiogenesis can be administered to the mice. In vivo host angiogenesis can be evaluated by means such as measuring the number of such cells within the graft, the density of such cells (cells/μm² or μm³), and the rate of their progression into the core of the graft. Use of alternate transgenes, for example, luciferase, green fluorescent protein or the like may permit more convenient evaluation of angiogenesis by allowing in-life, longitudinal measurement of host-derived endothelial cells within the graft. Use of alternate promoters, for example the promoter for lymphatic-specific genes such as FLT4, podoplanin, or the homeobox-containing gene Prox-1 to drive the transgene would permit similar evaluation of lymphangiogenesis. The same approach could be applied to promoters that are specific for genes associated with other processes that occur during the formation of adipose tissue.

Further, the methods of the present invention can be used to screen for agents that modulate the lymphangiogenic activity of adipocytes, preadipocytes, or adipose tissue. The agent identified could be used to modulate the development of new lymph vessels or the expansion of pre-existing lymph vessels. This process could include both the formation of small lymph vessels composed of a single layer of lymphatic endothelial cells (LECs) surrounded by an incomplete basement membrane and of larger lymph vessels, many of which are lined by lymphatic smooth muscle.

The methods of the present invention can further be used to identify agents that modulate the arteriogenic activity of adipocytes, preadipocytes, or adipose tissue. Agents can be identified agents which can stimulate or inhibit the development of larger blood vessels that supply a capillary bed (arterioles). Development may occur by means of increasing the blood carrying capacity of existing small arterioles, by creation of new arterioles from smaller blood vessels (non-arterioles), or by de novo generation of new arterioles. These physiological effects can be measured.

Cell populations contemplated for implantation in practicing methods of identifying agents that modulate the angiogenic, lymphangiogenic, and arteriogenic activity of adipose tissue, preadipocytes or adipocytes include populations identified based on their ability to differentiate into adipocytes, or to proliferate and differentiate into adipocytes, according to methods of the present invention. In some embodiments, the cell populations used to identify agents that modulate the angiogenic, lymphangiogenic, and arteriogenic activity of adipose tissue, preadipocytes or adipocytes are isolated as described in Examples IV and V, below. Briefly, the cell population can be obtained by obtaining isolated adipose-derived regenerative cells from a subject and sorting the adipose-derived regenerative cells into at least two different cell populations according to cell surface markers present on the cells. One or more of the subpopulations of sorted cells can be used for the screening methods described herein. See, e.g., Examples IV and V, below.

### G. Evaluation of Agents

Evaluation of candidate agents using any of the methods of the invention can be performed according to methods known to those of skill in the art. Modulation of a process can be assessed by comparing a rate or an absolute value of a parameter representative of adipocyte growth, health or differentiation obtained in the presence of a potential modulator with a value obtained in a control experiment. A control can be, e.g., nontreatment, treatment with an agent known to have no effect on a particular parameter, or treatment with an agent that produces a known effect on a given parameter. A control can also include the use of a different cell population for implantation. Additional controls can be identified by methods known to those of skill in the art.

For example, adipose tissue carrying a promoter/reporter gene construct as described herein can be processed to yield isolated adipocytes that carry the construct. These cells can be placed in a format consistent with high throughput screening, for example a 96 well plate, and exposed to candidate agents. The activity of candidate agents can be evaluated, e.g., by determination of their effect on expression of the reporter gene. Internal control genes can be included (such that the cells carry two constructs) so as to increase the assay's ability to discriminate between agents that non-specifically modulate expression of multiple genes and those that are specific for the gene of interest.

In some embodiments, the cells used in the methods of the present invention can be immortalized by means known in the art (for example, use of SV40 large T antigen or proliferation-associated genes in combination with telomerase). The immortalized cells can be screened to detect those capable of robust in vivo adipogenesis, for use in screening. For example, immortalized cells can be further modified to carry one or more promoter/reporter gene constructs. In one embodiment, the cells can be cloned to generate a homogeneous population that will provide increased reproducibility in screening. Adipogenic cells from an individual with a known genetic predisposition to obesity or with some other characteristics of interest, can be immortalized and used to generate adipose tissue in lipoatrophic host animals. The tissue, or adipocytes or other cells derived from this tissue, can then be used in drug screening as described herein. This approach is similar to the use of non-immortalized cells described herein but permits generation of a standardized reagent that could reduce variations in studies resulting from donor-to-donor differences. The use of conditionally-immortalized cells, i.e., cells that can be reversibly changed from a non-immortal to immortalized state by changing culture conditions or additives is also contemplated. Expression of transgenes that reversibly confer immortality, e.g., temperature-sensitive variants of simian virus 40 large T antigen, are known in the art. Methods of conferring conditional immortality are also known in the art and have been described in the literature.

Modulation of gene expression can be determined and/or measured, e.g., by quantitating the nucleic acid, e.g., RNA or cDNA, made from specific genes. In embodiments, the expression of a gene or protein is upregulated or downregulated at least 1.5-fold (e.g., 1.6-fold, 1.7-fold, 1.8-fold, 1.9-fold, 2-fold, 5-fold, 10-fold or more, or any amount in between), relative to the control gene expression. It will be understood by those of skill in the art that the modulatory activity of an agent can be identified through any of a number of comparisons of any of a number of parameters. For example, adipocyte proliferation can be measured in the presence and absence of an agent, and the measurements compared. Additionally, multiple timepoints can be obtained in the presence and absence of the agent, and the proliferation rates compared. Further, the effect of an agent can be compared with that of an agent having a known effect, to determine the difference in the effect of the agents on a parameter of interest.

### 7. Use of Modulating Agents for Treating Diseases and Disorders

### Adipocyte-Associated Conditions

Excess or insufficient adipose tissue is associated with a number of diseases and disorders. In embodiments, the methods of the present invention are used to screen for agents useful for treating such diseases. Such agents can be recombinant forms of adipokines or other molecules that affect the production or activity of adipokines. For example, the response to leptin has been reported to play a role in obesity, lipodystrophy, insulin resistance, dyslipidemia and amenorrhoea. Pathways involving adiponectin, an insulin-sensitizing factor, can be targeted to treat atherosclerosis, as well as insulin resistance, obesity, and dislypidemia. Similarly, pathways involving other adipokines, e.g., visfatin, RBP4 (retinol-binding protein 4), TNF-α, PAI-1 (plasminogen activator inhibitor-1), glucocorticoids (e.g., cortisol), can be targeted by potential agents to treat human disease. Adipokines and their associated disorders are discussed, e.g., by Klein, et al., 2006, Trends Endocrin. Metab. 17(1):26-32.

Specifically, agents that increase adipocyte or preadipocyte apoptosis or that inhibit the generation of new adipocytes can be used to treat obesity. The systemic inflammation associated with metabolic syndrome (also referred to as obesity metabolic syndrome) can be treated by agents that reduce adipocyte number, or that alter expression of immunomodulatory, pro-inflammatory, or anti-inflammatory molecules by adipose tissue, preadipocytes, or adipocytes.

Agents that promote adipogenesis can be used to treat lipoatrophy or lipodystrophy (for example the redistribution lipodystrophy that is frequently encountered in HIV-positive patients treated with highly active anti-retroviral therapy). Agents that modulate adipose-derived proinflammatory mediators can have a beneficial role in ameliorating the symptoms associated with metabolic syndrome in type 2 diabetes, and therefore be useful in the treatment of metabolic syndrome. Similarly, agents that modulate the angiogenic aspects of adipose tissue and adipocyte biology can be used to improve wound healing and the treatment of ischemic injury.

Recently it has been established that there are positive correlations between obesity and inflammatory disease, including inflammatory cardiovascular disease. The AZIP model can be used to study the relationship between developing adipose tissue, or adipose tissue that has been developed, for example under specific dietary conditions, and the recruitment and development of leukocytes either within the specific tissue or the organism as a whole. More specifically, the purpose would be to determine the molecular and biochemical mechanisms linking the adipose tissue's influence on the inflammatory behavior of the leukocyte pool, and vice versa. Also, the model could be used to study methods or materials to influence or control the interaction between the adipose tissue and the leukocytes, for example, in the search for a drug to control either inflammation or obesity. An example, of how to implement this model would be to introduce identifiable syngeneic leukocytes (sex mismatched or containing an isoform of a surface protein recognizable by flow cytometry and distinct from that of the host) from a donor animal into the adipose tissue bearing AZIP animal, and monitoring the development of the labeled leukocyte pool over time for markers of inflammatory cells. An example of a tool set to identify inflammatory cells could be the ex vivo measurement of inflammatory cytokine profiles (IFNγ, TNF, IL-17, etc) in the tissue resident or circulating leukocyte pool. An increase in production of these cytokines by activated leukocytes is indicative of an increase in the inflammatory status of the population.

### 8. Use of Adipocyte-Generating Cells for Soft Tissue Filling and Treatment of Diseases and Disorders

Cell populations identified using the methods of the present invention can be used to produce autologous or nonautologous soft tissue, or highly pure fat, for soft tissue implantation or regeneration applications known to those of skill in the art and described in the literature. Identification of relatively pure soft-tissue producing cell populations would allow production of a longer-lasting soft tissue using many fewer or no contaminating cells than are used in current methods, e.g., fat transplantation.

Soft tissue implantation can be useful for minimizing the appearance of or healing soft tissue defects, e.g., defects addressed using cosmetic procedures. Soft tissue implantation includes breast implantation, as well as implantation in or regeneration of any area of the body as deemed desirable by a patient or physician, e.g., filling or reshaping scars, wrinkles, pockmarks, etc. It further includes treatment of certain disorders, including stress urinary incontinence, oral gingival tissue defects, and defects that occur as a function of a surgical excision (resulting from, e.g., tumor removal, trauma, or cosmetic procedures). Soft tissue can also be used for intracordal injections of the laryngeal voice generator by changing the shape of this soft tissue mass.

### 9. Pharmaceutical Preparations

The dosage ranges for the administration of a therapeutic agent depend upon the type of agent and its potency. Ranges comprise amounts sufficient to produce the desired effect wherein the effect on the adipocyte-associated condition is favorable. The dosage should not be so large as to cause adverse side effects, such as hyperviscosity syndromes, pulmonary edema, congestive heart failure, etc., as such side effects may outweigh the benefits derived from the therapeutic agent. Generally, the dosage will vary with the age, condition, sex and extent of the disease in the patient and can be determined by one of skill in the art. The dosage can also be adjusted by the individual physician in the event of any complication.

Inhibition of symptoms can be measured according to methods described herein, or by other methods known to one skilled in the art. Methods for assessing the effect on an adipocyte-associated condition will depend on the condition being treated, and for the particular condition, such methods will be known to those of skill in the art.

It is to be appreciated that the potency, and therefore an expression of a “therapeutically effective” amount can vary. One skilled in the art can readily assess the potency of a gene product of this invention. Potency can be measured by a variety of means, all as described herein and in the literature and known to those of skill in the art, and the like assays.

A therapeutically effective amount of an agent for treating an adipose-associated condition can be determined by prevention or amelioration of adverse conditions or symptoms of diseases, injuries or disorders being treated. The appropriate dosage will of course vary depending upon, for example, the stage and severity of the disease or disorder to be treated and the mode of administration.

The therapeutic agents of the invention can be administered parenterally by injection or by gradual infusion over time. Although the tissue to be treated can typically be accessed in the body by systemic administration and therefore treated by intravenous administration of therapeutic compositions, other tissues and delivery means are contemplated where there is a likelihood that the tissue targeted contains the target molecule. Thus, therapeutic agents of the invention can be administered through the vasculature, intraperitoneally, orally, rectally, intramuscularly, subcutaneously, intracavity, transdermally, and can be delivered by peristaltic means.

Therapeutic compositions are conventionally administered intravenously, as by injection of a unit dose, for example. The term “unit dose” when used in reference to a therapeutic composition of the present invention refers to physically discrete units suitable as unitary dosage for the subject, each unit containing a predetermined quantity of active material calculated to produce the desired therapeutic effect in association with the required diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. Precise amounts of active ingredient required to be administered depend on the judgment of the practitioner and are peculiar to each individual. However, suitable dosage ranges for systemic application are disclosed herein and depend on the route of administration. Suitable regimes for administration are also variable, but are typified by an initial administration followed by repeated doses at one or more hour intervals by a subsequent injection or other administration. Alternatively, continuous intravenous infusion sufficient to maintain concentrations in the blood in the ranges specified for in vivo therapies are contemplated.

The present invention contemplates therapeutic compositions useful for practicing the therapeutic methods described herein. Therapeutic compositions of the present invention contain a physiologically tolerable carrier together with the therapeutic agent as described herein, dissolved or dispersed therein as an active ingredient. In a preferred embodiment, the therapeutic agent is not immunogenic when administered to a mammal or human patient for therapeutic purposes.

The invention further contemplates the administration of combinations of agents of the present invention, as well as combinations of these agents with other drugs or therapies, e.g., other drugs or treatments for diabetes.

As used herein, the terms “pharmaceutically acceptable,” “physiologically tolerable,” and grammatical variations thereof, as they refer to compositions, carriers, diluents and reagents, are used interchangeably and indicate that the materials are capable of administration to or upon a mammal without the production of undesirable physiological effects such as nausea, dizziness, gastric upset and the like.

The preparation of a pharmacological composition that contains active ingredients dissolved or dispersed therein is well understood in the art and need not be limited based on formulation. Typically such compositions are prepared as injectables either as liquid solutions or suspensions, however, solid forms suitable for solution, or suspensions in liquid prior to use can also be prepared. The preparation can also be emulsified.

The active ingredient can be mixed with excipients which are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient.

The therapeutic composition of the present invention can include pharmaceutically acceptable salts of the components therein. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic, etc. Salts formed with free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes.

Liquid compositions can also contain liquid phases in addition to and to the exclusion of water. Examples of such additional liquid phases are glycerin, vegetable oils such as cottonseed oil, and water-oil emulsions.

In further embodiments, the invention enables any of the foregoing methods to be carried out in combination with other therapies such as, for example, treatment with another compound, e.g., insulin or enbrel.

### 10. Patients

The invention contemplates treatment of patients including human patients. The term patient as used in the present application refers to all different types of mammals including humans and the present invention is effective with respect to all such mammals. The present invention is effective in treating any mammalian species having an adipocyte-associated disease or disorder, as described herein.

The contents of all cited references, including literature references, issued patents, published patent applications, and co-pending patent applications, cited throughout this application are hereby expressly incorporated by reference in their entirety.

### EXAMPLES

The present invention is further illustrated by the following examples, which should not be construed as limiting in any way.

### Example I

### Generation of De Novo Adipose Tissue from Adipocyte-depleted Adipose Tissue-Derived Cells

Adipose tissue was dissected from the inguinal region of nine FVB GFPU mice (Jackson Laboratories) aged 1-5 months. Blunt dissection was used to break the tissue into small fragments approximately 1-3 mm in diameter. Tissue fragments were digested at 37° C. with 0.075% Collagenase (Sigma Chemical Company) in PBS for 55 minutes with rocking. Following centrifugation and washing to remove mature adipocytes and residual tissue aggregates and connective tissue, cell number and viability were determined by dye exclusion. Viability was 93% as determined by co-staining with acridine orange and ethidium bromide and visualizing under a fluorescence microscope. Cells were resuspended in either phosphate-buffered saline (PBS), Matrigel, or a collagen gel at 1.6 million cells/mL.

One milliliter of cells (or an equal volume of cell-free vehicle only) was injected into both the dorsal and ventral subcutaneous space of 12 mice derived by crossing male mice, heterozygous for expression of the A-ZIP genotype, with wild-type FRVB females. Progeny expressing the A-ZIP genotype were identified by use of the polymerase chain reaction using primers specific for the A-ZIP transgene.

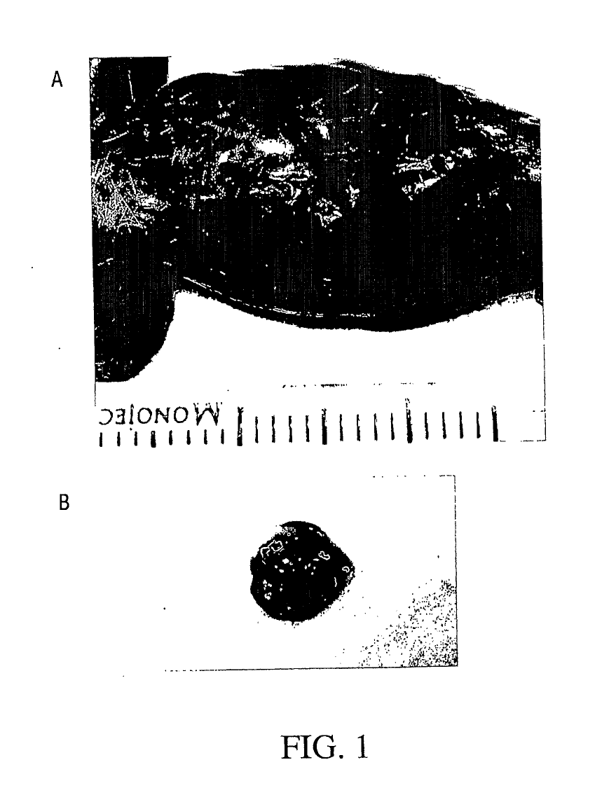

Seven weeks after injection one animal treated with Matrigel and cells was euthanized and dissected for recovery of implanted material. A small tissue mass was detected on the surface of the peritoneal muscle (FIG. 1). This tissue was dissected out and weighed (weight 0.134 g; total animal weight 24.7 g). Approximately one half of the tissue was prepared for histology by embedding material in OCT medium while the remainder was digested with 0.075% collagenase for 40 minutes. Digestion yielded 640,000 viable cells as determined by co-staining with acridine orange and ethidium bromide and quantitation using a hemocytometer under a fluorescence microscope.

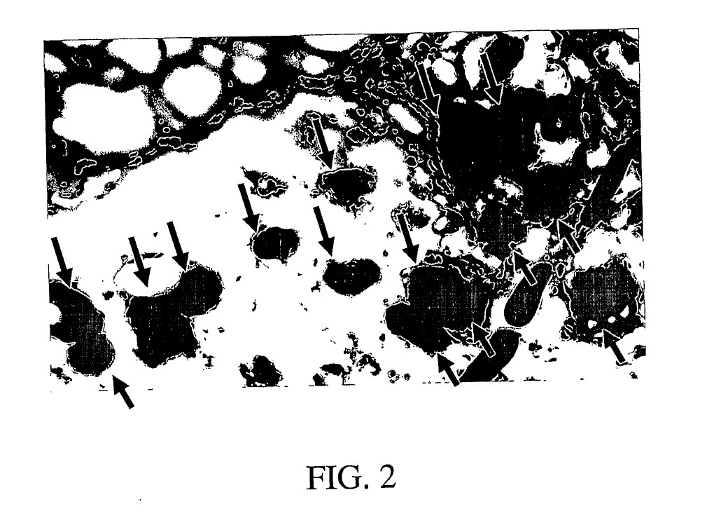

Staining with hematoxylin and eosin and with Oil Red O revealed that the implant was comprised of vascularized connective tissue with substantial regions of Oil Red O positivity (FIG. 2). Oil Red O is a standard stain used to detect adipocytes.

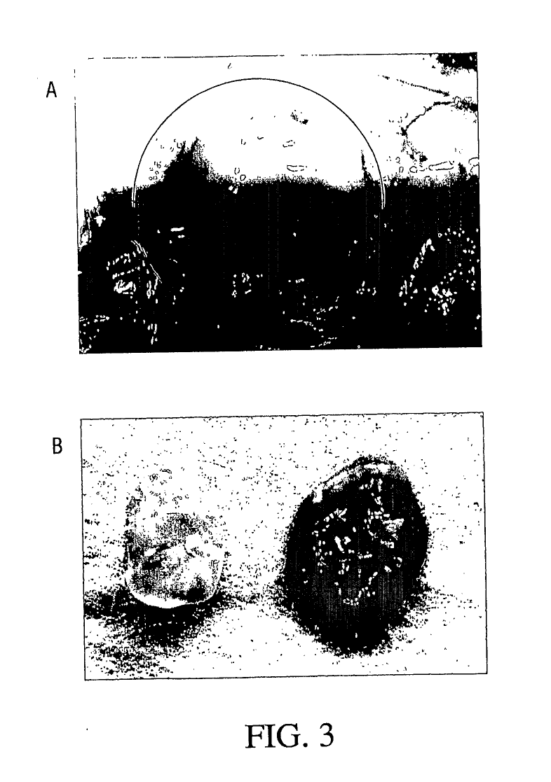

Remaining animals were euthanized between 12 and 14 weeks after cell injection. No implants were detected in animals treated with PBS alone or with PBS and cells. No implants were detected in animals receiving collagen gel alone. However, implants were observed in all animals treated with collagen gel supplemented with cells and in all animals receiving Matrigel although control (Matrigel only, no cells) implants appeared transparent while those that were generated from Matrigel supplemented with cells were opaque and white (see FIG. 3).

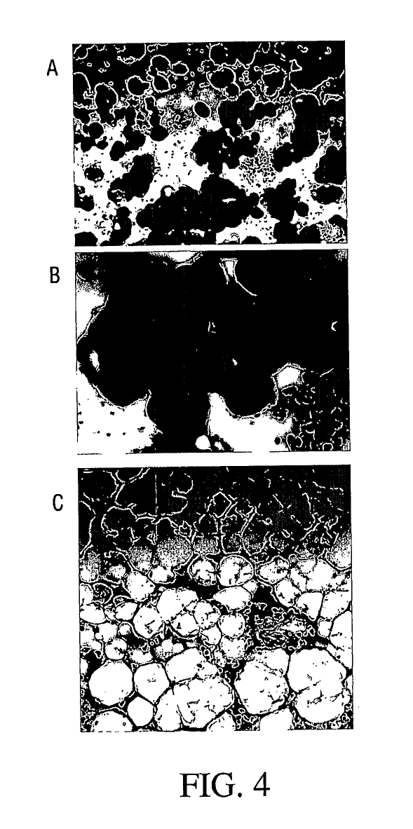

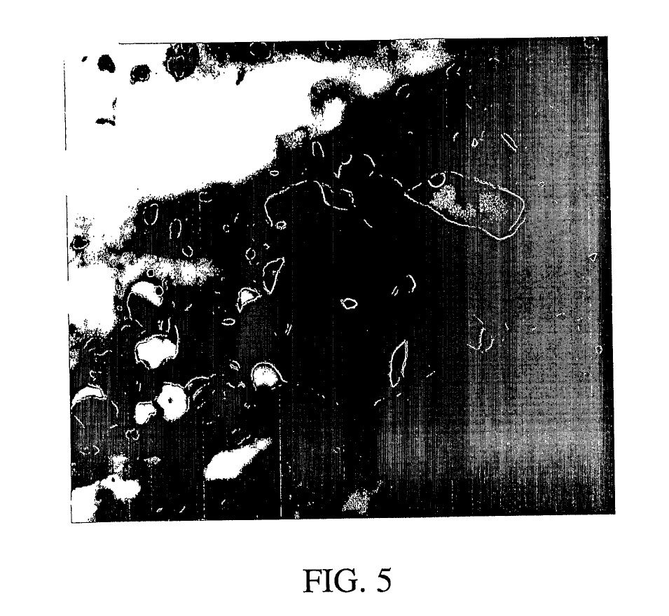

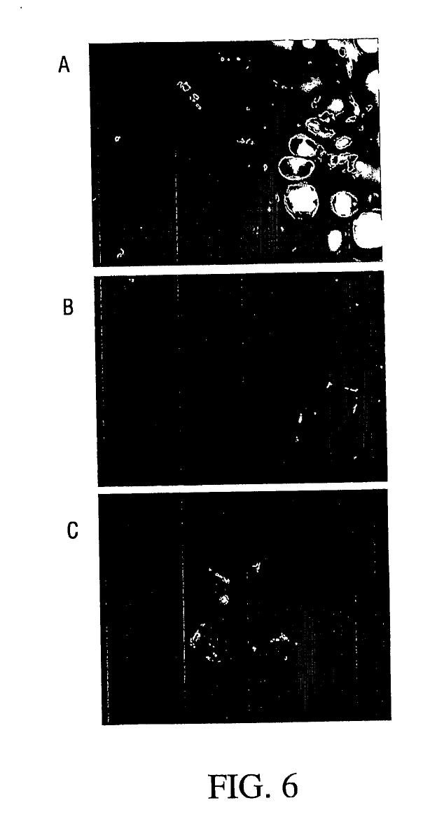

Histological evaluation showed considerable adipose tissue in cell-supplemented implants but not in cell-free controls. An example of tissue formation in Matrigel is shown in FIG. 4. An example of tissue formation in collagen gel is shown in FIG. 5. Examination under fluorescence microscopy revealed GFP expression within areas of the graft containing adipocytes (FIG. 6). Histologic evaluation of the matrigel-only, control implant revealed no regions of adipose tissue.

This example demonstrates that adipose-tissue derived cells can be used to generate adipose tissue in lipoatrophic hosts.

### Example II

### Generation of De Novo Adipose Tissue from Cultured Adipose Tissue-Derived Cells

Adipose tissue was dissected from the inguinal region of nine FVB GFPU mice (Jackson Laboratories) aged 1-5 months. Blunt dissection was used to break the tissue into small fragments approximately 1-3 mm in diameter. Tissue fragments were digested with 0.075% Collagenase (Sigma Chemical Company) for 55 minutes. Following centrifugation and washing to remove mature adipocytes and residual tissue aggregates and connective tissue cell number and viability were determined by dye exclusion. Cells were plated in tissue culture medium (DMEM/F12 supplemented with 10% fetal calf serum, and antibiotic/antimycotic solution). Cultures were fed with bi-weekly demi-depopulation and were passaged by trypsinization at approximately 80% confluence. After two passages cells at 50-80% confluence were harvested and resuspended in PBS, collagen gel, or matrigel. A-ZIP mice, generated as described above, were injected in the subcutaneous space of both flanks with 1.5 million cells (3 million cells/animal).

Approximately 10 weeks after injection animals were euthanized and implants dissected out. As in Example I animals receiving PBS (with or without cells) and animals receiving collagen gel alone (no cells) exhibited no implants. Control animals receiving Matrigel alone contained bilateral transparent implants. By contrast, animals receiving collagen gel and cultured cells and those receiving Matrigel and cultured cells exhibited opaque implants.

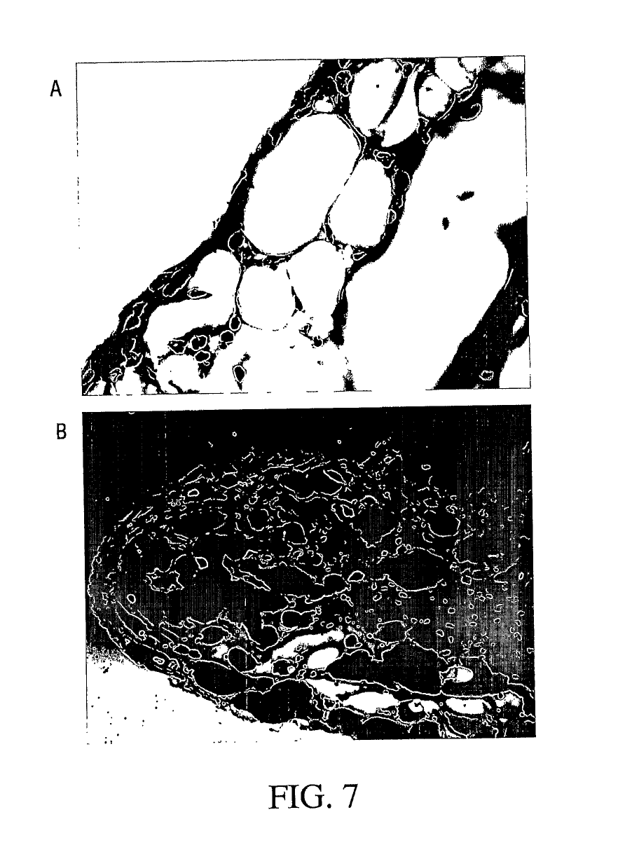

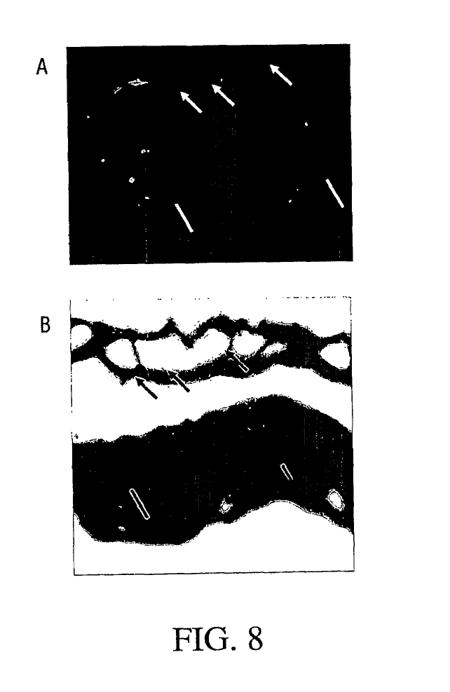

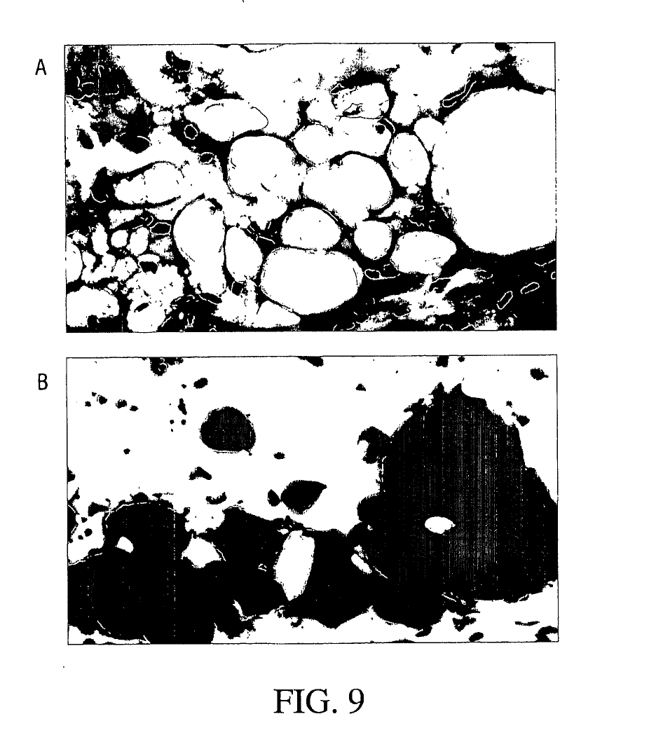

As with implants supplement with uncultured cells, histologic evaluation of Collagen-based implants showed generation of adipose tissue (FIG. 7). Fluorescence microscopy (FIG. 8) showed that areas of the implant containing adipocytes were fluorescent (arrows) while areas of fibrosis were not (lines) consistent with the donor origin of the adipose tissue. Similarly, implants comprised of matrigel supplemented with ADSC also gave rise to adipose tissue (FIG. 9).

This example demonstrates that adipose-tissue derived cells can be used as a source for the generation of adipose tissue in lipoatrophic hosts.

### Example III

### Generation of De Novo Adipose Tissue from Pre-Differentiated Cultured Adipose Tissue-Derived Cells

Adipose tissue-derived cells generated from donor cells in lipoatrophic mice are removed from the mice, placed in culture, and exposed to agents that induce in vitro differentiation towards adipocytes. Culture conditions known in the art and described in the literature are used, e.g., agents can include combinations of dexamethasone, insulin, and peroxisome proliferator-activated receptor gamma agonists. Cultured adipose tissue-derived cells exposed to these conditions for brief (less than 24 hours), intermediate (24 hours to 1 week), or prolonged (greater than 1 week) are harvested and combined with Matrigel, as described in Examples I and II, or seeded onto solid scaffolds such as those described herein. As described above, the cells are implanted into A-ZIP mice to generate adipose tissue.

### Example IV

### Use of an In Vivo Assay to Identify Murine ADRC Subpopulations with In Vivo Adipocyte Differentiation and Proliferation Capacity

The following experiments describe the isolation of a cell population that includes cells capable of differentiating into adipocytes, i.e., preadipocytes. The in vivo differentiation capabilities of specific adipose-derived cell subpopulations were evaluated using assay methods of the present invention.

Cells were obtained from GFP-transgenic FVB mice obtained from Jackson Laboratories (Bar Harbor, Me.). Tissue was removed from the inguinal fat pad following euthanasia and processed as described in Example I. During dissection care was taken to eliminate lymph nodes. For this study 15 million cells were obtained from 20 donor animals.

First, the cells were stained with antibodies to CD45 (coupled to the fluorochrome APC), CD90 (coupled to the fluorochrome PE), and Sca-1 (coupled to the fluorochrome PECy7). Cells were sorted into the three populations shown in Table 1 using a FACSAria™ Cell Sorting System and FACSDiva™ software (both from Becton Dickinson). The sorted cells were implanted in a lipoatrophic (A-ZIP/F1) mouse, and the in vivo ability of the cells in each subpopulation to develop into mature adipocytes and proliferate into cells that could develop into adipocytes was assessed.

The three cell populations sorted/isolated by flow cytometry as described above were resuspended in complete medium and diluted 1:1 with Matrigel. The CD45⁻/Sca-1⁻ sorting yielded 47,721 cells at 99.6% purity, the CD45⁻/Sca-1⁺/CD90^{low} sorting yielded 38,055 cells at 92.8% purity (2.4% were CD45⁻/Sca-1⁺/CD90⁺), and the CD45⁻/Sca-1⁺/CD90⁺ yielded 109,083 cells at 98.1% purity.

Each isolated population was injected in two aliquots into each of two high glucose (0.400 g/dL) A-ZIP/F1 mice (0.5 ml/implant). Recipient mice were euthanized at nine weeks, and grafts were retrieved and prepared for histology by embedding in OCT. Frozen sections were stained with hematoxylin and eosin (H & E) or Oil Red O (ORO).

TABLE 1
Histological analysis of CD45⁻ subpopulations - 1

| Sorted Subpopulation | Oil Red O | Clustering |
|---|---|---|
| CD45⁻/Sca-1⁻ | + | − |
| CD45⁻/Sca-1⁺/CD90^{low} | + | + |
| CD45⁻/Sca-1⁺/CD90⁺ | + | +/− |

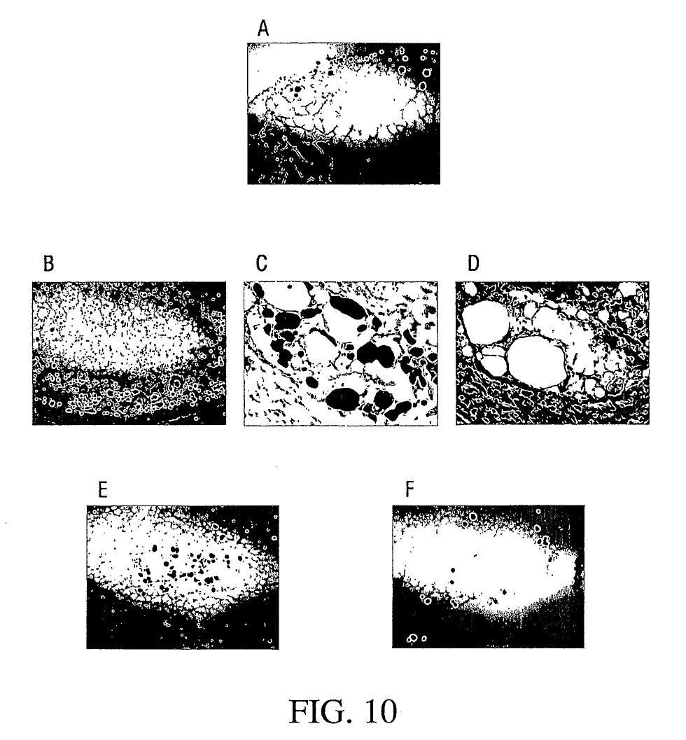

FIG. 10 shows adipocyte (ORO) and nuclear (H & E) staining in the grafts from animals that received the CD45⁻/Sca-1⁻ cell subpopulation. These grafts were not observed to contain many ORO-stained cells, and those that were observed were for the most part not present in clusters.

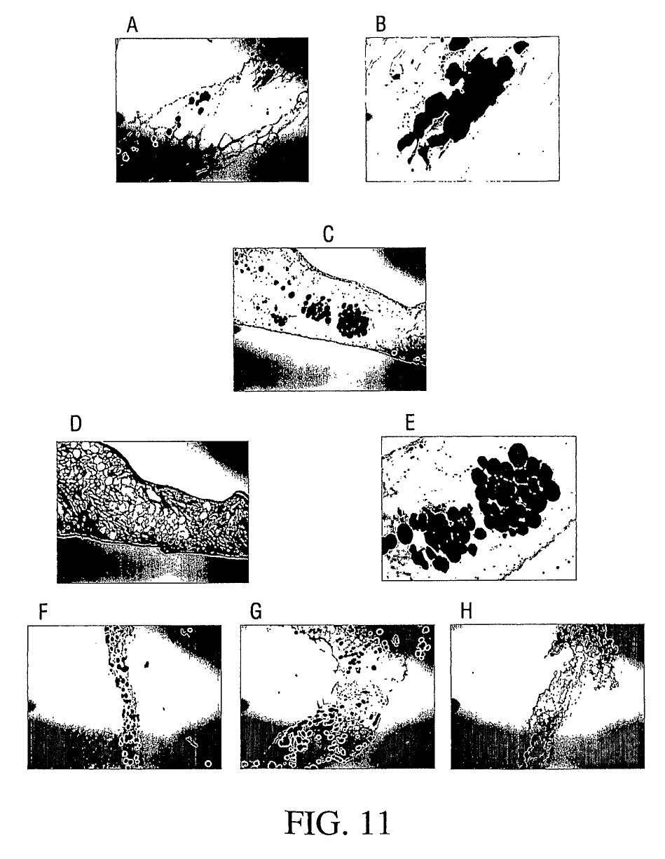

The images in FIG. 11 show ORO staining of the CD45⁻/Sca-1⁺/CD90⁻ subpopulation grafts. In general, these grafts were observed to have many ORO stained cells present in tight clusters.

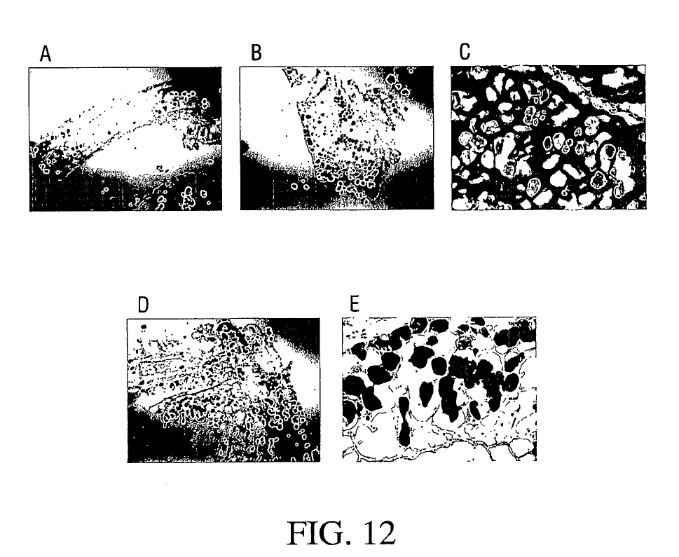

FIG. 12 shows staining of the grafts that arose from implantation of the CD45⁻/Sca-1⁺/CD90⁺ subpopulation. This subpopulation produced many scattered ORO-stained cells, with a small number of clusters. ORO-positive cells appear dark. Adipocytes in H & E appear as transparent or light gray areas that are roughly circular. Clusters appear as transparent or light gray areas with a honeycomb-like outline.

Table 1 summarizes the findings for each of the CD45⁻ subpopulations described above. As shown in the Figures and described in Table 1, the ORO-staining cells in the two Sca-1⁺ populations were observed as small clusters, indicating adipocyte proliferation. In contrast, the Sca-1⁻ cells did not form clusters.

In the next experiment, performed in a similar manner, the cells were stained with additional markers: CD45 (coupled to the fluorochrome APC-Cy7); Sca-1 (coupled to the fluorochrome PE-Cy7); CD90 (coupled to the fluorochrome PerCP Cy5); CD31 (coupled to the fluorochrome APC); and CD73 (coupled to the fluorochrome PE). The sorted populations used in this experiment are shown in Table 2.

We observed that the CD45⁻/Sca-1⁺/CD31⁻ population was separated into two populations on the basis of CD90 and CD73 expression. One, referred to herein as CD90⁺, expressed high levels of CD90 such that the fluorescence intensity of the population was greater than that of the isotype control. The second population, referred to herein as CD90^{low}, expressed considerably less CD90 such that there was substantial overlap in the CD90 fluorescence intensity of this population and that of the isotype control. Therefore, a threshold level to separate the cells that are truly CD90-negative (exhibit absence of CD90) from those that express low levels of this marker was not determined. This second population is referred to as CD90^{low}.

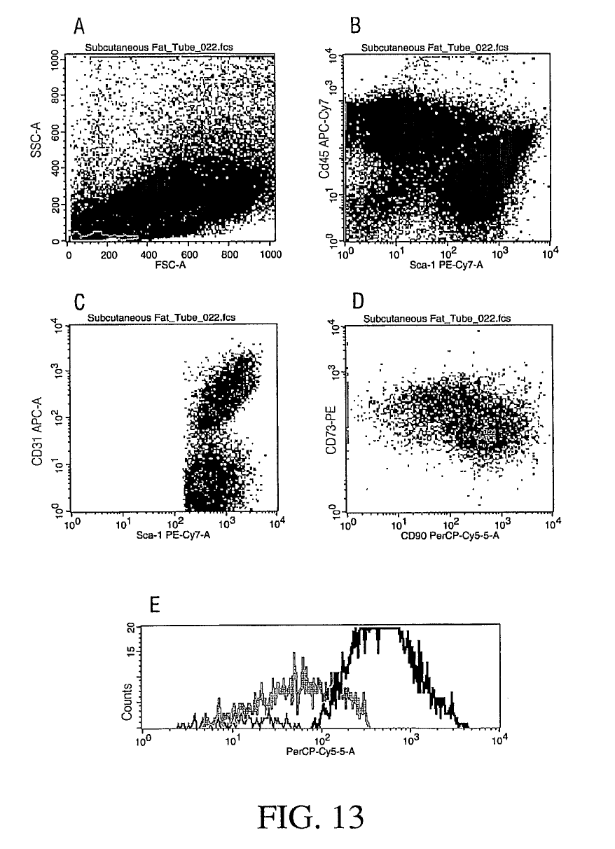

CD90⁺ cells exhibited substantially lower fluorescence intensity for CD73 than the CD90^{low} cells. That is, cells expressing low levels of CD90 expressed high levels of CD73 whereas cells with high levels of CD90 showed little or no expression of CD73. These two populations are referred to herein as CD45⁻/Sca-1⁺/CD31⁻/CD90^{low}/CD73⁺ and CD45⁻/Sca-1⁺/CD31⁻/CD90⁺/CD73⁻. FIG. 13 shows the gating strategy and CD90 fluorescence intensity profiles of the CD45⁻/Sca-1⁺/CD31⁻/CD90^{low}/CD73⁺ population, along with comparison with the isotype control for CD90 for this population and with the CD90 expression profile of the CD45⁻/Sca-1⁺/CD31⁻/CD90⁺/CD73⁻ population.

CD45⁻/Sca-1⁺/CD31⁻/CD90^{low}/CD73⁺ sorted cells (84% purity) were implanted at two cell concentrations: 1,000 sorted cells or 10,000 sorted cells per implant. CD45⁻/Sca-1⁺/CD31⁻/CD90⁺/CD73⁻ sorted cells (84% purity) were implanted at 10,000 cells per implant. CD45⁺/Sca-1⁺ cells (94% purity) were implanted at 5,000 cells per implant and CD45⁻/Sca-1⁺ cells (86% purity) were implanted at 10,000 cells per implant. The grafts were assayed at two months post-implantation for adipocyte differentiation and proliferation as described above. Both doses of CD45⁻/Sca-1⁺/CD31⁻/CD90^{low}/CD73⁺ sorted cells gave rise to clusters of adipocytes.

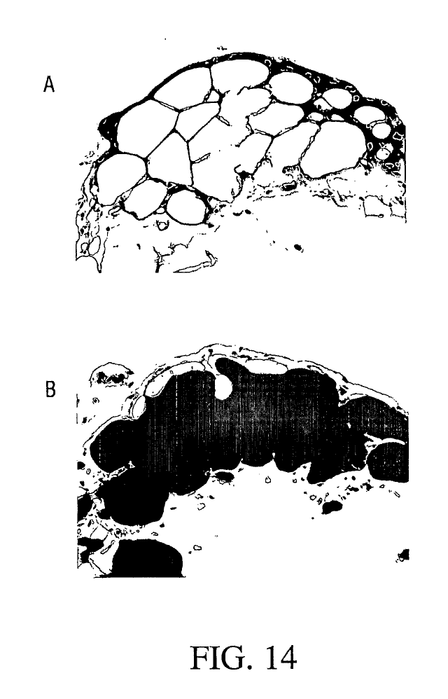

FIG. 14 shows Oil Red O staining of the cells from a graft made using 10,000 CD45⁻/Sca-1⁺/CD31⁻/CD90^{low}/CD73⁺ sorted cells.

No clustered adipocytes were observed in grafts generated from implantation of 10,000 CD45⁻/Sca⁺/CD31⁻/CD90⁺/CD73⁻ sorted cells. Scattered Oil Red O positive cells (none in clusters) were observed in grafts generated from implantation of CD45⁺/Sca-1⁺ cells. As demonstrated in the previous study, clusters of Oil Red O-positive cells were observed in grafts generated from implantation of CD45⁻/Sca-1⁺ cells.

These results demonstrate that the murine cell population, CD45⁻/Sca-1⁺/CD31⁻/CD90^{low}/CD73⁺ is capable of proliferating and generating adipocytes, whereas the CD45⁻/Sca-1⁺/CD31⁻/CD90⁺/CD73⁻ cells do not.

TABLE 2
Histological analysis of CD45⁻ subpopulations - 2

| Sorted Subpopulation | Oil Red O | Clustering |
|---|---|---|
| CD45⁻/Sca-1⁺ | + | + |
| CD45^{-/}Sca-1⁺/CD31⁻/CD90^{low}/CD73⁺ | + | + |
| CD45⁻/Sca-1⁺/CD31⁻/CD90⁺/CD73⁻ | + | − |
| CD45⁺/Sca-1⁺ | + | − |

Accordingly, adipose-derived regenerative cells isolated by flow cytometry into a subpopulation of, CD45⁻/Sca-1⁺/CD31⁻/CD90^{low}/CD73⁺ cells can be used to generate adipocytes. These purified populations can also be used in several other approaches to generate tissue.

### Example V

### Identification of a Human Cell Population with Surface Phenotype CD45⁻/CD31⁻/CD90^{low}/CD73⁺

Based on the results described above, the murine ADRC population CD45⁻/Sca-1⁺/CD31⁻/CD90^{low}/CD73⁺ is capable of proliferation and adipocyte generation in vivo. The experiments described below demonstrate the identification of CD45⁻/CD31⁻/CD90^{low}/CD73⁺ from human ADRCs, as assessed by flow cytometry.

Lipoaspirate obtained by suction assisted lipoplasty (liposuction) following informed patient consent was washed with Lactated Ringer's solution, to remove excess blood and tumescent solution, and digested with a collagenase-containing enzyme solution. The digestate was centrifuged to concentrate a cell pellet and washed to remove residual enzyme. The cells were then stained with an antibody panel similar to that used above for murine cells. Using this approach we observed a population of human cells that had the cell surface phenotype CD45⁻/CD31⁻/CD90^{low}/CD73⁺. Cells within the CD45⁻/CD31⁻/CD90^{low}/CD73⁺ population were CD34⁺, and did not express CD146, CD105, or CD13.

### Example VI

### Generation of De Novo Adipose Tissue from Cultured Marrow-Derived Cells

To generate adipose tissue from cultured marrow derived cells, bone marrow is extracted from the femurs of FVB GFPU mice (Jackson Laboratories) aged 1-5 months. Briefly, following carbon dioxide-mediated euthanasia the hind limb is dissected out and muscle and lower limb (foot) are dissected yielding a clean femur. The distal head of the femur is severed and a 21 G needle is inserted through the cartilage endplate of the proximal end of the bone into the medullary cavity. Tissue culture medium (DMEM/F12 supplemented with antibiotics and 10% fetal calf serum) is then perfused through the medullary cavity expelling a plug of red marrow into a receptacle. The marrow is then gently triturated through a 16 G needle to yield a single cell suspension that is filtered through a 75 μm filter. Cells are then plated at a cell density of 20-40×10⁶ cells per 9.5 cm². After 72 hours non-adherent cells are discarded and the adherent layer is rinsed with fresh medium and refed. Cultures are then re-fed twice weekly and passaged at 70-80% of confluence for 3-4 weeks. Cells are harvested by trypsinization, resuspended in matrigel, and injected into the subcutaneous space of both flanks of A-ZIP (1.5 million cells/injection; 3 million cells/animal). Approximately 10 weeks after injection animals are euthanized and implants dissected out and evaluated for adipogenesis using Hematoxylin and Eosin staining and Oil Red O staining as described above.

### Example VII

### Generation of De Novo Adipose Tissue on Solid Scaffolds

Adipose tissue is generated from donor cells (e.g., CD45⁻/CD31⁻/CD90^{low}/CD73⁺ subpopulations of cells isolated as described in Examples IV-V) using solid phase support scaffolds. Sponge-like polyglycolide (PGA) scaffolds are washed, sterilized, and seeded with cells (fresh cells, cultured cells, or cultured/predifferentiated cells) that are implanted by injection or surgical insertion or other means known in the art or described herein. This approach provides a solid substrate to which the cells can attach and proliferate and/or differentiate.

In separate experiments, cell-seeded implants are supplemented by loading the cells in Matrigel or by coating the scaffold with Matrigel or other agents prior to implantation.

### Example VIII

### Extraction of Preadipocytes from De Novo Generated Adipose Tissue

Preadipocytes are extracted from tissue generated as described in Example I. As discussed therein, approximately one half of the implant yielded 640,000 non-buoyant cells. This population contains cells that do not contain sufficient lipid to be buoyant and therefore do not appear to be mature adipocytes. Rather, the population includes preadipocytes, cells committed to adipocytic differentiation that have not yet matured or differentiated into adipocytes.

### Example IX

### Extraction of Adipocytes from De Novo Generated Adipose Tissue

The implants are retrieved and digested to obtain newly-generated adipocytes that contain the genotype of the donor cells. As shown in the histology described above, the implants contained lipid-laden adipocytes. The methods used to generate the implants are such that few, if any, such cells were originally implanted. Thus, the adipocytes detected in the implants are newly formed. This is consistent with their size, amount of lipid content, and exhibition of green fluorescence, a characteristic of the donor animal. As the recipient animals are genetically incapable of generating adipocytes and do not express green fluorescent protein, the adipocytes were donor-derived.

### Example X

### Adipocyte Apoptosis

Human adipose tissue-derived cells are prepared, placed in culture, and exposed to a retrovirus carrying a selectable marker and the reporter gene luciferase under the control of the Bak gene promoter. Using standard molecular biology and cell culture techniques, stable transfectants are isolated and injected into the subcutaneous, intramuscular, intraperitoneal, or other competent space of immunotolerant, lipoatrophic mice.

Following sufficient time to allow development of adipose tissue from the donor cells the implants are dissected out and the tissue digested with collagenase to release the cells from connective tissue. Mature adipocytes are harvested on the basis of their buoyancy or by other means known to those of skill in the art and placed in culture. These cells are then exposed to candidate agents. The ability to induce or inhibit adipocyte apoptosis is assessed by measuring the induction or repression of luciferase expression. In vivo screening of candidates is also achieved by treating mice with candidate agents. The induction or repression of luciferase expression is measured by either invasive or non-invasive means known to those skilled in the art.

### Example XI

### Screening for Agents that Modulate Adipogenesis and Adipotoxicity

Agents that Modulate Adipogenesis

Adipose tissue-derived cells are prepared and injected into the subcutaneous space of lipoatrophic mice as described in Examples I and II. Animals are treated with a candidate agent to assess the candidate compound's ability to modulate adipogenesis. Following administration of the candidate agent to the animal, leptin production from the implanted cell population is measured using standard techniques (e.g., gene expression or immunoblotting). An increase or decrease in leptin production in the implanted cells is detected, and is indicative of the compound's ability to modulate adipogenesis. Alternatively, the implanted tissue is harvested. The number of adipocytes in the harvested tissue is assessed by staining hematoxylin and eosin (H & E) or Oil Red O (ORO) as described in Examples I and II.

Agents that Modulate Adipotoxicity

Adipose tissue-derived cells from humans are prepared and injected into the subcutaneous space of lipoatrophic mice as described in Examples I and II. The mice are incapable of mounting an effective immune response to the implanted material. This results in the generation in vivo of adipose tissue composed of human cells.

Candidate compounds are administered to the host animal. Gene expression, histochemical, and/or immunological assays described herein are used to measure the presence of adipocytes and adipose tissue arising from the implanted cells. Agents which modulate human adipogenesis or adipo-toxicity in vivo are identified.

Agents that Differentially Affect Various Adipose Depots

Adipose tissue is isolated from different fat depots, e.g., visceral and subcutaneous, using the methods described in Examples I-III. Adipose-derived cells are isolated as described herein. The cells are implanted into lipoatrophic mice as described herein. The animal is administered a candidate compound and adipogenesis is measured as described above. The ability of candidate compounds to modulate adipogenesis from tissue derived from different depots is determined.

Identification of Markers that are Differentially Expressed in Adipose Tissue from Various Depots

Adipose tissue is isolated from different fat depots as described in Examples I-III. Adipose derived cells are isolated from the adipose tissue. The cells are implanted into lipoatrophic mice as described herein. The adipose tissue derived from the implant is excised as described in Examples I-II. Expression of cell surface markers or other markers are measured, (e.g., by FACs analysis, gene arrays, etc.) to determine the gene expression profile of the cells from the excised tissue, using routine molecular biology techniques.

Candidate compounds are also tested for their ability to convert the phenotype of one depot (for example, visceral adipose) to that of another (for example, subcutaneous adipose).

### Example XII

### Adipose Angiogenesis

The present invention is applied to evaluate angiogenesis and to screen for agents capable of modulating angiogenesis. Lipoatrophic A-ZIP mice are cross-bred with animals that are transgenic for a marker gene, FVB/N-Tg(TIE2-lacZ)182Sato/J mice available from Jackson Laboratories. This cross-breeding creates a strain of mouse that is lipoatrophic and in which the lacZ transgene is expressed exclusively in endothelial cells. Adipose tissue-derived cells are prepared from wild-type FVB mice and injected into the subcutaneous or intraperitoneal space of A-ZIP/Tie2lacZ lipoatrophic mice as described above. The vasculature of the newly-formed adipose tissue formed thereby includes host-derived endothelial cells. In vivo host angiogenesis is evaluated by measuring the number of host-derived endothelial cells within the graft, the density of these cells (cells/μm² or μm³), and the rate of their progression into the core of the graft.

1. A method for identifying an isolated population of adipose-derived regenerative cells capable of generating adipocytes or adipose tissue in a subject, comprising:
a. obtaining isolated adipose-derived regenerative cells from a subject;
b. sorting said isolated adipose-derived regenerative cells into at least two different cell populations according to cell surface markers present on said cells;
c. providing at least one of said at least two different cell populations to at least one host animal; and
d. determining the presence, absence, quality, or amount of adipocytes or adipose tissue generated by the at least one of said two different cell populations provided in step (c) in said at least one host animal.

2. A method for identifying a molecule that modulates a biological property of adipocytes or adipose tissue in a subject, comprising:
a. obtaining isolated adipose-derived regenerative cells from a subject;
b. providing said isolated adipose-derived regenerative cells to at least one host animal;
c. determining the presence, absence, quality, or amount of adipocytes or adipose tissue generated by said isolated adipose-derived regenerative cells in said at least one host animal;
d. providing a candidate molecule that modulates a biological property of adipocytes or adipose tissue to said host animal; and
e. determining whether said candidate molecule modulates a biological property of adipocytes or adipose tissue in said host animal.

3. A method for identifying a molecule that modulates the activity of a toxicant on adipocytes or adipose tissue in a subject, comprising:
a. obtaining isolated adipose-derived regenerative cells from a subject;
b. providing said isolated adipose-derived regenerative cells to at least one host animal;
c. determining the presence, absence, quality, or amount of adipocytes or adipose tissue generated by said isolated adipose-derived regenerative cells in said at least one host animal;
d. providing said toxicant to said at least one host animal;
e. providing a candidate molecule that modulates the activity of a toxicant on adipocytes or adipose tissue to said host animal; and
f. determining whether said candidate molecule modulates the activity of a toxicant on adipocytes or adipose tissue in said host animal.

4. A method of making an adipose-derived regenerative cell medicament comprising:
a. obtaining isolated adipose-derived regenerative cells from a subject;
b. sorting said isolated adipose-derived regenerative cells into at least two different cell populations according to cell surface markers present on said cells;
c. providing at least one of said at least two different cell populations to at least one host animal;
d. determining the presence, absence, quality, or amount of adipocytes or adipose tissue generated by the at least one of said two different cell populations provided in step (c) in said at least one host animal; and
e. incorporating a cell population that is determined to generate adipocytes or adipose tissue in step (d) into a medicament.

5. A method of adipose-derived regenerative cell transplantation comprising:
a. obtaining isolated adipose-derived regenerative cells from a subject;
b. sorting said isolated adipose-derived regenerative cells into at least two different cell populations according to cell surface markers present on said cells;
c. providing at least one of said at least two different cell populations to at least one host animal;
d. determining the presence, absence, quality, or amount of adipocytes or adipose tissue generated by the at least one of said two different cell populations provided in step (c) in said at least one host animal;
e. incorporating a cell population that is determined to generate adipocytes or adipose tissue in step (d) into a medicament; and
f. providing said medicament to a patient that is identified as one in need of adipose-derived regenerative cell transplantation.

6. The method of claim 1, wherein the host animal or subject or both are immunotolerant, syngenic, or lipoatropic.

7. The method of claim 1, wherein the presence, absence, quality, or amount of adipocytes or adipose tissue generated by at least two different cell populations sorted according to cell surface markers are compared in either the same or different host animals.

8. The method of claim 1, wherein in a first model, the presence, absence, quality, or amount of adipocytes or adipose tissue generated by at least one of the at least two different cell populations sorted according to cell surface markers is compared to a second model, wherein the presence or absence of adipocytes or adipose tissue generated by the isolated adipose-regenerative cells prior to cell sorting in either the same or different host animals are determined.

9. The method of claim 2, wherein said isolated adipose-derived regenerative cells are sorted into at least two different cell populations according to cell surface markers present on said cells and at least one of said at least two different sorted cell populations are provided to at least one host animal to which the candidate molecule or the candidate molecule and toxicant are provided.

10. The method of claim 3, wherein said isolated adipose-derived regenerative cells are sorted into at least two different cell populations according to cell surface markers present on said cells and at least two different sorted cell populations are provided to at least one host animal to which the candidate molecule or the candidate molecule and toxicant are provided, and, optionally, the modulation of the biological property of adipocytes or adipose tissue or the modulation of the activity of the toxicant at the sites of introduction of said at least two different sorted cell populations are compared.

11. The method of claim 2, wherein in a first model, said isolated adipose-derived regenerative cells are sorted into at least two different cell populations according to cell surface markers present on said cells, at least one of the at least two different sorted cell populations are provided to at least one host animal to which the candidate molecule or the candidate molecule and toxicant are provided and in a second model, a portion of said isolated adipose-derived regenerative cells are provided to either the same or a different host animal to which the candidate molecule or the candidate molecule and toxicant are provided, and, optionally, the modulation of the biological property of adipocytes or adipose tissue or the modulation of the activity of the toxicant in the two models are compared.

12. The method of claim 1, wherein the isolated adipose-derived regenerative cells are from a human.

13. The method of claim 1, wherein the host animal is a human.

14. The method of claim 1, wherein the host animal is a mouse.

15. The method of claim 1, wherein the subject from which the isolated adipose-derived regenerative cells are obtained and host animal, which receives said isolated adipose-derived regenerative cells are the same species.

16. The method of claim 1, wherein the subject from which the isolated adipose-derived regenerative cells are obtained and host animal, which receives said isolated adipose-derived regenerative cells are the same individual.

17. The method of claim 1, wherein the isolated adipose-derived regenerative cells and/or a sorted cell population are genetically modified prior to providing said the isolated adipose-derived regenerative cells and/or a sorted cell population to said host animal.

18. The method of claim 17, wherein said isolated adipose-derived regenerative cells and/or a sorted cell population is genetically modified with a marker gene such as, GFP, luciferase, or B-gal.

19. The method of claim 1, wherein said isolated adipose-derived regenerative cells and/or a sorted cell population are isolated while maintaining a closed/sterile fluid pathway.

20. The method of claim 1, wherein said sorting utilizes flow cytometry.

21. The method of claim 1, wherein said sorting is based on analysis of at least two cell surface markers, at least three cell surface markers, at least four cell surface markers, at least five cell surface markers, or at least six cell surface markers.

22. The method of claim 1, wherein said at least two different cell populations are provided to the same host animal at different locations.

23. The method of claim 1, wherein said at least two different cell populations are provided to different host animals of the same species.

24. The method of claim 1, wherein said at least two different cell populations are provided to different host animals of different species.

25. The method of claim 1, wherein the presence or absence of adipocytes or adipose tissue in said at least one host animal is determined by measuring the appearance, size, morphology, or a biochemical marker of said adipocytes or adipose tissue.

26. The method of claim 1, further comprising determining the presence, absence, quality, or amount of angiogenesis, arteriogenesis, or lymphangiogenesis in said host animal.

27. The method of claim 26, wherein said determination of the presence, absence, quality, or amount of adipocytes or adipose tissue in said at least one host animal is determined by histology.

28. The method of claim 26, wherein said determination of the presence, absence, quality, or amount of adipocytes or adipose tissue in said at least one host animal is determined by staining.

29. The method of claim 26, wherein said determination of the presence, absence, quality, or amount of adipocytes or adipose tissue in said at least one host animal is determined by detection of a biological marker without sacrificing the host animal.

30. The method of claim 26, wherein said determination of the presence, absence, quality, or amount of adipocytes or adipose tissue in said at least one host animal is determined by detection of a GFP without sacrificing the host animal.

31. The method of claim 1, wherein at least one of the two different cell populations that are provided to at least one host animal expresses CD73, does not express CD45 or CD31, and expresses low levels of or no CD90.

32. The method of claim 2, wherein said modulation of activity is an up-regulation of activity.

33. The method of claim 3, wherein said modulation of activity is a down-regulation of activity.

34. The method of claim 2, wherein said candidate molecule is a hormone, an adipokine, an angiogenic modulating molecule, a lymphangiogenic modulating molecule, an immunomodulatory molecule, or an arteriogenic modulatory molecule.

35. The method of claim 5, wherein said patient that is identified as one in need of adipose-derived regenerative cell transplantation is a patient in need or that desires soft tissue implantation or regeneration.

36. The method of claim 35, wherein the cell population provided to said patient expresses CD73, does not express CD45 or CD31, and that expresses low levels of CD90 or no CD90.

37. The method of claim 5, wherein said patient that is identified as one in need of adipose-derived regenerative cell transplantation is a patient with obesity, obesity metabolic syndrome, or diabetes.

38. The method of claim 37, wherein the cell population provided to said patient expresses CD73, does not express CD45 or CD31, and that expresses low levels of CD90 or no CD90.

39. The method of claim 5, wherein said patient that is identified as one in need of adipose-derived regenerative cell transplantation is a patient with a cardiovascular disorder or peripheral vascular disease.

40. The method of claim 39, wherein the cell population provided to said patient expresses CD73, does not express CD45 or CD31, and that expresses low levels of CD90 or no CD90.

41. (canceled)
